SEQUENCE LISTING (38 sequences)

SEQ ID NO: 1 - protein, 740 aa, Streptococcus pneumoniae
MGWSHPQFEKASVKDTYTDRLDDWNGIIAGNQYYDSKNDQMAKLNQELEGKVADSLSSISSQADRIYLWEKFSNYKTSANLTATYRKLEEMAKQVTNPSSRYYQDETVVRTVRDSMEWMHKHVYNSEKSIVGNWWDYEIGTPRAINNTLSLMKEYFSDEEIKKYTDVIEKFVPDPEHFRKTTDNPFKALGGNLVDMGRVKVIAGLLRKDDQEISSTIRSIEQVFKLVDQGEGFYQDGSYIDHTNVAYTGAYGNVLIDGLSQLLPVIQKTKNPIDKDKMQTMYHWIDKSFAPLLVNGELMDMSRGRSISRANSEGHVAAVEVLRGIHRIADMSEGETKQRLQSLVKTIVQSDSYYDVFKNLKTYKDISLMQSLLSDAGVASVPRTSYLSAFNKMDKTAMYNAEKGFGFGLSLFSSRTLNYEHMNKENKRGWYTSDGMFYLYNGDLSHYSDGYWPTVNPYKMPGTTETDAKRADSDTGKVLPSAFVGTSKLDDANATATMDFTNWNQTLTAHKSWFMLKDKIAFLGSNIQNTSTDTAATTIDQRKLESSNPYKVYVNDKEASLTEQEKDYPETQSGFLESSDSKKNIGYFFFKKSSISMSKALQKGAWKDINEGQSDKEVENEFLTISQAHKQNGDSYGYMLIPNVDRATFNQMIKELESSLIENNETLQSVYDAKQGVWGIVKYDDSVSTISNQFQVLKRGVYTIRKEGDEYKIAYYNPETQESAPDQEVFKKLEHHHHHH

SEQ ID NO: 2 - DNA, 2251 bp, Streptococcus pneumoniae
  source 1..2251: mol_type = genomic DNA
ccatgggctggagccatccgcagtttgaaaaagcaagcgtgaaggatacgtacacggaccgcttagatgactggaacgggatcatcgcggggaaccagtactatgatagcaaaaacgaccaaatggcaaaattaaatcaagaattagaaggtaaggtcgcggattcactctcaagcattagcagccaagctgaccggatctatttatgggaaaaatttagcaactataaaacaagcgccaaccttacggcaacgtatcgaaaattagaggagatggcaaagcaggtaacgaacccgagcagccgctattatcaggatgaaacggtcgtgcggacggtccgagattcaatggaatggatgcataaacatgtctacaacagcgaaaagtcaattgtggggaattggtgggattatgaaatcggcacgccgcgcgcaatcaataacacgttaagccttatgaaagaatacttcagcgatgaggaaattaaaaaatatacggatgtaattgaaaaatttgtcccagatccagaacatttccggaagacaacggataatccgttcaaggcgctcggcggtaatttagtggatatgggtcgagtcaaagtcatagcgggcttacttcgcaaggatgatcaggaaattagcagcacgattcggtcaattgagcaggtattcaagttagtcgaccagggcgaaggcttttatcaggatggttcatatatcgaccacacgaacgtggcatatacaggcgcgtatgggaacgtgttaattgatgggttgagccagcttttaccggtcattcagaagacgaagaacccgatcgataaagataaaatgcagacgatgtaccactggattgataaatcatttgcgccgttacttgtaaacggtgagcttatggatatgagccgcggtcggtcaatcagccgcgccaactcagaggggcacgtagcagccgtcgaagtcctccgggggattcaccggatagctgatatgagcgaaggtgaaacgaaacagcgcttacaaagcttagtaaagacgattgtgcagtcagatagctattatgatgtctttaagaacttaaagacgtataaggatatcagcttaatgcagtcattattaagcgatgccggtgtcgccagcgtgccgcggacgagctacctcagcgcatttaataagatggataaaacggcaatgtacaacgccgagaaagggtttggttttgggttaagcttgttttcaagccgcacgttaaactacgaacacatgaataaggaaaacaaacgcggctggtatacaagcgatgggatgttctatttatacaacggggatttaagccactattcagatgggtactggccgacggtgaacccgtataagatgccgggcacgacggagacagatgcgaagcgggctgattcagatacgggcaaagtgttacccagcgcgttcgtgggtacatcaaaactcgatgatgcaaacgctacggccacgatggatttcacgaattggaaccagacgttaacggcgcataagtcatggtttatgctcaaggataagattgcatttttaggttcaaacatccagaatacgagcacggatacggcggccacgacgattgaccaacggaaacttgaaagcagcaacccgtataaagtctatgtcaacgataaagaagcatcattaacggaacaggaaaaggattatccggaaacgcagagcgggtttttagaatcatcagattcaaaaaagaacattggctactttttctttaagaagagcagcatcagcatgagcaaggcgttacagaagggtgcatggaaggatatcaacgaaggtcaaagcgacaaggaagtggaaaacgaatttttaacaattagccaagcgcataagcagaacggtgatagctatgggtatatgttgattccgaatgtagatcgcgcaacgttcaaccagatgataaaagagttagaatcatcattgatcgaaaacaatgaaacgttacaaagcgtgtatgatgcaaaacagggtgtgtgggggattgtaaaatatgatgatagcgtcagcacgatttcaaatcagttccaggtgttaaaacgcggtgtctatacgattcggaaagaaggggatgaatataagattgcatactataacccggaaacgcaagaaagcgcgccggatcaagaagtctttaaaaagctcgagcatcatcatcatcatcactaaccactcgacttcttttggagctgagc

SEQ ID NO: 3 - protein, 949 aa, Streptococcus pneumoniae
MVPIEAKKKYKLRFKIKTDNKVGIAKVRIIEESGKDKRLWNSATTSGTKDWQTIEADYSPTLDVDKIKLELFYETGTGTVSFKDIELVEVADQPSEDSQTDKQLEEKIDLPIGKKHVFPLADYTYKVENPDVASVKNGILEPLKEGTTNVIVSKDGKEVKKIPLKILASVKDTYTDRLDDWNGIIAGNQYYDSKNDQMAKLNQELEGKVADSLSSISSQADRIYLWEKFSNYKTSANLTATYRKLEEMAKQVTNPSSRYYQDETVVRTVRDSMEWMHKHVYNSEKSIVGNWWDYEIGTPRAINNTLSLMKEYFSDEEIKKYTDVIEKFVPDPEHFRKTTDNPFKALGGNLVDMGRVKVIAGLLRKDDQEISSTIRSIEQVFKLVDQGEGFYQDGSYIDHTNVAYTGAYGNVLIDGLSQLLPVIQKTKNPIDKDKMQTMYHWIDKSFAPLLVNGELMDMSRGRSISRANSEGHVAAVEVLRGIHRIADMSEGETKQRLQSLVKTIVQSDSYYDVFKNLKTYKDISLMQSLLSDAGVASVPRTSYLSAFNKMDKTAMYNAEKGFGFGLSLFSSRTLNYEHMNKENKRGWYTSDGMFYLYNGDLSHYSDGYWPTVNPYKMPGTTETDAKRADSDTGKVLPSAFVGTSKLDDANATATMDFTNWNQTLTAHKSWFMLKDKIAFLGSNIQNTSTDTAATTIDQRKLESSNPYKVYVNDKEASLTEQEKDYPETQSGFLESSDSKKNIGYFFFKKSSISMSKALQKGAWKDINEGQSDKEVENEFLTISQAHKQNGDSYGYMLIPNVDRATFNQMIKELESSLIENNETLQSVYDAKQGVWGIVKYDDSVSTISNQFQVLKRGVYTIRKEGDEYKIAYYNPETQESAPDQEVFKKLEQAAQPQVQNSKEKEKSEEEKNHSDQKNLPQTGEGQSILASLGFLLLGAFYLFRRGKNN

SEQ ID NO: 4 - DNA, 2850 bp, Streptococcus pneumoniae
  source 1..2850: mol_type = genomic DNA
atggttcctattgaagctaagaaaaagtataaactgcgtttcaagattaaaacagataataaagtcgggattgccaaagttcgtatcattgaggaaagtggtaaggacaagcgattgtggaattctgcaacgacgtcaggaacaaaggactggcagaccattgaagcagactatagcccgactttagatgttgataaaatcaagctggagttattctatgaaacaggaactgggactgtttcctttaaggatattgagctggtagaggtagcagaccagccttctgaggattctcaaacagataaacaacttgaggaaaagattgatttaccaattggaaaaaaacatgtttttcctcttgcggactatacttataaggtagaaaatcctgacgttgcttcagtcaaaaatggaattttagaacctcttaaggaagggacaaccaatgtcattgtcagtaaagatggcaaggaagtgaaaaagattcctttgaagattctagcctctgttaaggatacatacacagaccgtttggatgactggaatggcatcatcgctgggaatcaatactatgattctaaaaatgaccagatggccaaattaaaccaggaattggaaggaaaggtagctgatagcctatccagtatttcaagtcaggcggaccgcatctatttgtgggaaaaattttcaaattataaaacgtctgcaaatctgactgccacttatcggaaattggaggagatggccaagcaagtgaccaatccttcttctcgttattatcaagatgaaactgtcgttcgaacagtcagggattccatggaatggatgcataaacatgtctacaatagtgaaaagagcattgttgggaactggtgggattatgaaatcggtacacctcgtgccatcaacaataccttgtctctgatgaaagaatacttctctgatgaggaaattaaaaaatatacagatgtgattgaaaaatttgtaccagatcccgaacatttccgaaagacgactgataacccattcaaggctctaggtggaaacttagttgatatgggaagggtaaaagtaatagctggtttactgcgtaaggatgatcaagaaatttcttctaccattcgctcgattgagcaagtgttcaagttggtagaccaaggtgaaggtttttatcaagatggatcctatatcgaccacaccaatgttgcctatacgggtgcttatgggaatgttttgattgatggcctgtctcaactgttgccagtcattcaaaagaccaagaatccaatcgataaagataaaatgcaaaccatgtaccactggattgataaatcgtttgctcctttgctggtgaatggagagctgatggatatgagtcgtggacgctcgatcagtcgtgcaaatagcgaggggcacgtggccgcagtagaagtactaagagggattcaccgaatagcggatatgtctgaaggagaaaccaaacaacgtttgcagagtcttgtgaagaccattgttcaatcggatagttattatgatgtctttaagaatttgaagacttataaggatatcagtttgatgcaatccttgttaagtgatgcaggagtcgcaagtgttccaagaacaagttacctatctgcctttaacaagatggataaaacagccatgtacaatgcagagaaagggtttggatttggcttgtcactcttttccagtcgtaccttgaattacgaacacatgaacaaggaaaataaacgtggttggtatacgagtgatgggatgttctatctttacaatggcgatttgagtcactatagcgatggctactggccaacagttaatccatataagatgcctggtacaacagagacggatgctaagagagcggatagcgatacaggtaaagttttaccgtctgctttcgttggaacgagcaaactagatgatgccaatgcgacagcaaccatggatttcaccaactggaatcaaacattgactgctcataagagctggtttatgctaaaggataagattgcctttttaggaagcaatatccaaaacacttcaacagatactgctgcaactacaattgaccagagaaaactggaatcaagtaatccatataaagtctatgtcaatgataaagaagcctcccttacagaacaagaaaaggattatcctgaaacccaaagtgggtttttagaatcgtccgattcgaaaaagaatattggttactttttctttaagaagagttcaatcagtatgagtaaggctttgcaaaagggagcctggaaggatatcaatgaaggacagtcagacaaggaagttgaaaatgaatttcttacgattagtcaggctcataagcaaaatggagattcttatggctatatgctcattcctaacgtggatcgtgccaccttcaatcaaatgataaaagagttagaaagcagcctcatcgaaaataacgaaacccttcagtctgtttatgatgccaaacaaggagtttggggcattgtgaaatatgatgattctgtctctactatttccaaccaattccaagttttgaaacgtggagtctatactattcgaaaagaaggggatgaatataagattgcctactataatcctgaaacccaggaatcagctccagatcaggaagtctttaaaaagctagagcaagcagctcagccacaagtacagaattcaaaagaaaaggaaaaatctgaagaggaaaagaaccattcggatcaaaagaatctccctcagacaggagaaggtcagtcaatcttggcaagtctagggttcttgctacttggggcgttttatttattccgtagaggaaagaacaactaa

SEQ ID NO: 5 - protein, 8 aa, synthetic construct
WSHPQFEK

SEQ ID NO: 6 - DNA, 24 bp, synthetic construct
  source 1..24: mol_type = other DNA
tggagccatccgcagtttgaaaaa

SEQ ID NO: 7 - protein, 6 aa, synthetic construct
HHHHHH

SEQ ID NO: 8 - DNA, 18 bp, synthetic construct
  source 1..18: mol_type = other DNA
catcatcatcatcatcac

SEQ ID NO: 9 - protein, 724 aa, Streptococcus pneumoniae
ASVKDTYTDRLDDWNGIIAGNQYYDSKNDQMAKLNQELEGKVADSLSSISSQADRIYLWEKFSNYKTSANLTATYRKLEEMAKQVTNPSSRYYQDETVVRTVRDSMEWMHKHVYNSEKSIVGNWWDYEIGTPRAINNTLSLMKEYFSDEEIKKYTDVIEKFVPDPEHFRKTTDNPFKALGGNLVDMGRVKVIAGLLRKDDQEISSTIRSIEQVFKLVDQGEGFYQDGSYIDHTNVAYTGAYGNVLIDGLSQLLPVIQKTKNPIDKDKMQTMYHWIDKSFAPLLVNGELMDMSRGRSISRANSEGHVAAVEVLRGIHRIADMSEGETKQRLQSLVKTIVQSDSYYDVFKNLKTYKDISLMQSLLSDAGVASVPRTSYLSAFNKMDKTAMYNAEKGFGFGLSLFSSRTLNYEHMNKENKRGWYTSDGMFYLYNGDLSHYSDGYWPTVNPYKMPGTTETDAKRADSDTGKVLPSAFVGTSKLDDANATATMDFTNWNQTLTAHKSWFMLKDKIAFLGSNIQNTSTDTAATTIDQRKLESSNPYKVYVNDKEASLTEQEKDYPETQSGFLESSDSKKNIGYFFFKKSSISMSKALQKGAWKDINEGQSDKEVENEFLTISQAHKQNGDSYGYMLIPNVDRATFNQMIKELESSLIENNETLQSVYDAKQGVWGIVKYDDSVSTISNQFQVLKRGVYTIRKEGDEYKIAYYNPETQESAPDQEVFKKLE

SEQ ID NO: 10 - protein, 734 aa, Streptococcus pneumoniae
MGWSHPQFEKASVKDTYTDRLDDWNGIIAGNQYYDSKNDQMAKLNQELEGKVADSLSSISSQADRIYLWEKFSNYKTSANLTATYRKLEEMAKQVTNPSSRYYQDETVVRTVRDSMEWMHKHVYNSEKSIVGNWWDYEIGTPRAINNTLSLMKEYFSDEEIKKYTDVIEKFVPDPEHFRKTTDNPFKALGGNLVDMGRVKVIAGLLRKDDQEISSTIRSIEQVFKLVDQGEGFYQDGSYIDHTNVAYTGAYGNVLIDGLSQLLPVIQKTKNPIDKDKMQTMYHWIDKSFAPLLVNGELMDMSRGRSISRANSEGHVAAVEVLRGIHRIADMSEGETKQRLQSLVKTIVQSDSYYDVFKNLKTYKDISLMQSLLSDAGVASVPRTSYLSAFNKMDKTAMYNAEKGFGFGLSLFSSRTLNYEHMNKENKRGWYTSDGMFYLYNGDLSHYSDGYWPTVNPYKMPGTTETDAKRADSDTGKVLPSAFVGTSKLDDANATATMDFTNWNQTLTAHKSWFMLKDKIAFLGSNIQNTSTDTAATTIDQRKLESSNPYKVYVNDKEASLTEQEKDYPETQSGFLESSDSKKNIGYFFFKKSSISMSKALQKGAWKDINEGQSDKEVENEFLTISQAHKQNGDSYGYMLIPNVDRATFNQMIKELESSLIENNETLQSVYDAKQGVWGIVKYDDSVSTISNQFQVLKRGVYTIRKEGDEYKIAYYNPETQESAPDQEVFKKLE

SEQ ID NO: 11 - protein, 12 aa, synthetic construct
MGSAWSHPQFEK

SEQ ID NO: 12 - protein, 6 aa, synthetic construct
ENLYFQ

SEQ ID NO: 13-14 - skipped (2 entries)

SEQ ID NO: 15 - DNA, 18 bp, synthetic construct
  source 1..18: mol_type = other DNA
gaaaacttgtatttccag

SEQ ID NO: 16 - protein, 6 aa, synthetic construct
ENLYFQ

SEQ ID NO: 17 - DNA, 15 bp, synthetic construct
  source 1..15: mol_type = other DNA
gatgatgatgataaa

SEQ ID NO: 18 - protein, 4 aa, synthetic construct
DDDK

SEQ ID NO: 19 - DNA, 2256 bp, Streptococcus pneumoniae
  source 1..2256: mol_type = genomic DNA
ccatgggcagcgcgtggagccatccgcagtttgaaaaagcaagcgtgaaggatacgtacacggaccgcttagatgactggaacgggatcatcgcggggaaccagtactatgatagcaaaaacgaccaaatggcaaaattaaatcaagaattagaaggtaaggtcgcggattcactctcaagcattagcagccaagctgaccggatctatttatgggaaaaatttagcaactataaaacaagcgccaaccttacggcaacgtatcgaaaattagaggagatggcaaagcaggtaacgaacccgagcagccgctattatcaggatgaaacggtcgtgcggacggtccgagattcaatggaatggatgcataaacatgtctacaacagcgaaaagtcaattgtggggaattggtgggattatgaaatcggcacgccgcgcgcaatcaataacacgttaagccttatgaaagaatacttcagcgatgaggaaattaaaaaatatacggatgtaattgaaaaatttgtcccagatccagaacatttccggaagacaacggataatccgttcaaggcgctcggcggtaatttagtggatatgggtcgagtcaaagtcatagcgggcttacttcgcaaggatgatcaggaaattagcagcacgattcggtcaattgagcaggtattcaagttagtcgaccagggcgaaggcttttatcaggatggttcatatatcgaccacacgaacgtggcatatacaggcgcgtatgggaacgtgttaattgatgggttgagccagcttttaccggtcattcagaagacgaagaacccgatcgataaagataaaatgcagacgatgtaccactggattgataaatcatttgcgccgttacttgtaaacggtgagcttatggatatgagccgcggtcggtcaatcagccgcgccaactcagaggggcacgtagcagccgtcgaagtcctccgggggattcaccggatagctgatatgagcgaaggtgaaacgaaacagcgcttacaaagcttagtaaagacgattgtgcagtcagatagctattatgatgtctttaagaacttaaagacgtataaggatatcagcttaatgcagtcattattaagcgatgccggtgtcgccagcgtgccgcggacgagctacctcagcgcatttaataagatggataaaacggcaatgtacaacgccgagaaagggtttggttttgggttaagcttgttttcaagccgcacgttaaactacgaacacatgaataaggaaaacaaacgcggctggtatacaagcgatgggatgttctatttatacaacggggatttaagccactattcagatgggtactggccgacggtgaacccgtataagatgccgggcacgacggagacagatgcgaagcgggctgattcagatacgggcaaagtgttacccagcgcgttcgtgggtacatcaaaactcgatgatgcaaacgctacggccacgatggatttcacgaattggaaccagacgttaacggcgcataagtcatggtttatgctcaaggataagattgcatttttaggttcaaacatccagaatacgagcacggatacggcggccacgacgattgaccaacggaaacttgaaagcagcaacccgtataaagtctatgtcaacgataaagaagcatcattaacggaacaggaaaaggattatccggaaacgcagagcgggtttttagaatcatcagattcaaaaaagaacattggctactttttctttaagaagagcagcatcagcatgagcaaggcgttacagaagggtgcatggaaggatatcaacgaaggtcaaagcgacaaggaagtggaaaacgaatttttaacaattagccaagcgcataagcagaacggtgatagctatgggtatatgttgattccgaatgtagatcgcgcaacgttcaaccagatgataaaagagttagaatcatcattgatcgaaaacaatgaaacgttacaaagcgtgtatgatgcaaaacagggtgtgtgggggattgtaaaatatgatgatagcgtcagcacgatttcaaatcagttccaggtgttaaaacgcggtgtctatacgattcggaaagaaggggatgaatataagattgcatactataacccggaaacgcaagaaagcgcgccggatcaagaagtctttaaaaagctcgaggaaaacttgtatttccagcatcatcatcatcatcactaagctgagc

SEQ ID NO: 20 - protein, 742 aa, Streptococcus pneumoniae
MGSAWSHPQFEKASVKDTYTDRLDDWNGIIAGNQYYDSKNDQMAKLNQELEGKVADSLSSISSQADRIYLWEKFSNYKTSANLTATYRKLEEMAKQVTNPSSRYYQDETVVRTVRDSMEWMHKHVYNSEKSIVGNWWDYEIGTPRAINNTLSLMKEYFSDEEIKKYTDVIEKFVPDPEHFRKTTDNPFKALGGNLVDMGRVKVIAGLLRKDDQEISSTIRSIEQVFKLVDQGEGFYQDGSYIDHTNVAYTGAYGNVLIDGLSQLLPVIQKTKNPIDKDKMQTMYHWIDKSFAPLLVNGELMDMSRGRSISRANSEGHVAAVEVLRGIHRIADMSEGETKQRLQSLVKTIVQSDSYYDVFKNLKTYKDISLMQSLLSDAGVASVPRTSYLSAFNKMDKTAMYNAEKGFGFGLSLFSSRTLNYEHMNKENKRGWYTSDGMFYLYNGDLSHYSDGYWPTVNPYKMPGTTETDAKRADSDTGKVLPSAFVGTSKLDDANATATMDFTNWNQTLTAHKSWFMLKDKIAFLGSNIQNTSTDTAATTIDQRKLESSNPYKVYVNDKEASLTEQEKDYPETQSGFLESSDSKKNIGYFFFKKSSISMSKALQKGAWKDINEGQSDKEVENEFLTISQAHKQNGDSYGYMLIPNVDRATFNQMIKELESSLIENNETLQSVYDAKQGVWGIVKYDDSVSTISNQFQVLKRGVYTIRKEGDEYKIAYYNPETQESAPDQEVFKKLEENLYFQ

SEQ ID NO: 21 - DNA, 2028 bp, Streptococcus pneumoniae
  source 1..2028: mol_type = genomic DNA
ccatgggcagcgcgtggagccatccgcagtttgaaaaagcaagcgtgaaggatacgtacacggaccgcttagatgactggaacgggatcatcgcggggaaccagtactatgatagcaaaaacgaccaaatggcaaaattaaatcaagaattagaaggtaaggtcgcggattcactctcaagcattagcagccaagctgaccggatctatttatgggaaaaatttagcaactataaaacaagcgccaaccttacggcaacgtatcgaaaattagaggagatggcaaagcaggtaacgaacccgagcagccgctattatcaggatgaaacggtcgtgcggacggtccgagattcaatggaatggatgcataaacatgtctacaacagcgaaaagtcaattgtggggaattggtgggattatgaaatcggcacgccgcgcgcaatcaataacacgttaagccttatgaaagaatacttcagcgatgaggaaattaaaaaatatacggatgtaattgaaaaatttgtcccagatccagaacatttccggaagacaacggataatccgttcaaggcgctcggcggtaatttagtggatatgggtcgagtcaaagtcatagcgggcttacttcgcaaggatgatcaggaaattagcagcacgattcggtcaattgagcaggtattcaagttagtcgaccagggcgaaggcttttatcaggatggttcatatatcgaccacacgaacgtggcatatacaggcgcgtatgggaacgtgttaattgatgggttgagccagcttttaccggtcattcagaagacgaagaacccgatcgataaagataaaatgcagacgatgtaccactggattgataaatcatttgcgccgttacttgtaaacggtgagcttatggatatgagccgcggtcggtcaatcagccgcgccaactcagaggggcacgtagcagccgtcgaagtcctccgggggattcaccggatagctgatatgagcgaaggtgaaacgaaacagcgcttacaaagcttagtaaagacgattgtgcagtcagatagctattatgatgtctttaagaacttaaagacgtataaggatatcagcttaatgcagtcattattaagcgatgccggtgtcgccagcgtgccgcggacgagctacctcagcgcatttaataagatggataaaacggcaatgtacaacgccgagaaagggtttggttttgggttaagcttgttttcaagccgcacgttaaactacgaacacatgaataaggaaaacaaacgcggctggtatacaagcgatgggatgttctatttatacaacggggatttaagccactattcagatgggtactggccgacggtgaacccgtataagatgccgggcacgacggagacagatgcgaagcgggctgattcagatacgggcaaagtgttacccagcgcgttcgtgggtacatcaaaactcgatgatgcaaacgctacggccacgatggatttcacgaattggaaccagacgttaacggcgcataagtcatggtttatgctcaaggataagattgcatttttaggttcaaacatccagaatacgagcacggatacggcggccacgacgattgaccaacggaaacttgaaagcagcaacccgtataaagtctatgtcaacgataaagaagcatcattaacggaacaggaaaaggattatccggaaacgcagagcgggtttttagaatcatcagattcaaaaaagaacattggctactttttctttaagaagagcagcatcagcatgagcaaggcgttacagaagggtgcatggaaggatatcaacgaaggtcaaagcgacaaggaagtggaaaacgaatttttaacaattagccaagcgcataagcagaacggtgatagctatgggtatatgttgattccgaatgtagatcgcgcaacgttcaaccagatgataaaagagttagaatcagaaaacttgtatttccagcatcatcatcatcatcactaagctgagc

SEQ ID NO: 22 - protein, 666 aa, Streptococcus pneumoniae
MGSAWSHPQFEKASVKDTYTDRLDDWNGIIAGNQYYDSKNDQMAKLNQELEGKVADSLSSISSQADRIYLWEKFSNYKTSANLTATYRKLEEMAKQVTNPSSRYYQDETVVRTVRDSMEWMHKHVYNSEKSIVGNWWDYEIGTPRAINNTLSLMKEYFSDEEIKKYTDVIEKFVPDPEHFRKTTDNPFKALGGNLVDMGRVKVIAGLLRKDDQEISSTIRSIEQVFKLVDQGEGFYQDGSYIDHTNVAYTGAYGNVLIDGLSQLLPVIQKTKNPIDKDKMQTMYHWIDKSFAPLLVNGELMDMSRGRSISRANSEGHVAAVEVLRGIHRIADMSEGETKQRLQSLVKTIVQSDSYYDVFKNLKTYKDISLMQSLLSDAGVASVPRTSYLSAFNKMDKTAMYNAEKGFGFGLSLFSSRTLNYEHMNKENKRGWYTSDGMFYLYNGDLSHYSDGYWPTVNPYKMPGTTETDAKRADSDTGKVLPSAFVGTSKLDDANATATMDFTNWNQTLTAHKSWFMLKDKIAFLGSNIQNTSTDTAATTIDQRKLESSNPYKVYVNDKEASLTEQEKDYPETQSGFLESSDSKKNIGYFFFKKSSISMSKALQKGAWKDINEGQSDKEVENEFLTISQAHKQNGDSYGYMLIPNVDRATFNQMIKELESENLYFQ

SEQ ID NO: 23 - DNA, 1989 bp, Streptococcus pneumoniae
  source 1..1989: mol_type = genomic DNA
ccatgggcagcgcgtggagccatccgcagtttgaaaaagcaagcgtgaaggatacgtacacggaccgcttagatgactggaacgggatcatcgcggggaaccagtactatgatagcaaaaacgaccaaatggcaaaattaaatcaagaattagaaggtaaggtcgcggattcactctcaagcattagcagccaagctgaccggatctatttatgggaaaaatttagcaactataaaacaagcgccaaccttacggcaacgtatcgaaaattagaggagatggcaaagcaggtaacgaacccgagcagccgctattatcaggatgaaacggtcgtgcggacggtccgagattcaatggaatggatgcataaacatgtctacaacagcgaaaagtcaattgtggggaattggtgggattatgaaatcggcacgccgcgcgcaatcaataacacgttaagccttatgaaagaatacttcagcgatgaggaaattaaaaaatatacggatgtaattgaaaaatttgtcccagatccagaacatttccggaagacaacggataatccgttcaaggcgctcggcggtaatttagtggatatgggtcgagtcaaagtcatagcgggcttacttcgcaaggatgatcaggaaattagcagcacgattcggtcaattgagcaggtattcaagttagtcgaccagggcgaaggcttttatcaggatggttcatatatcgaccacacgaacgtggcatatacaggcgcgtatgggaacgtgttaattgatgggttgagccagcttttaccggtcattcagaagacgaagaacccgatcgataaagataaaatgcagacgatgtaccactggattgataaatcatttgcgccgttacttgtaaacggtgagcttatggatatgagccgcggtcggtcaatcagccgcgccaactcagaggggcacgtagcagccgtcgaagtcctccgggggattcaccggatagctgatatgagcgaaggtgaaacgaaacagcgcttacaaagcttagtaaagacgattgtgcagtcagatagctattatgatgtctttaagaacttaaagacgtataaggatatcagcttaatgcagtcattattaagcgatgccggtgtcgccagcgtgccgcggacgagctacctcagcgcatttaataagatggataaaacggcaatgtacaacgccgagaaagggtttggttttgggttaagcttgttttcaagccgcacgttaaactacgaacacatgaataaggaaaacaaacgcggctggtatacaagcgatgggatgttctatttatacaacggggatttaagccactattcagatgggtactggccgacggtgaacccgtataagatgccgggcacgacggagacagatgcgaagcgggctgattcagatacgggcaaagtgttacccagcgcgttcgtgggtacatcaaaactcgatgatgcaaacgctacggccacgatggatttcacgaattggaaccagacgttaacggcgcataagtcatggtttatgctcaaggataagattgcatttttaggttcaaacatccagaatacgagcacggatacggcggccacgacgattgaccaacggaaacttgaaagcagcaacccgtataaagtctatgtcaacgataaagaagcatcattaacggaacaggaaaaggattatccggaaacgcagagcgggtttttagaatcatcagattcaaaaaagaacattggctactttttctttaagaagagcagcatcagcatgagcaaggcgttacagaagggtgcatggaaggatatcaacgaaggtcaaagcgacaaggaagtggaaaacgaatttttaacaattagccaagcgcataagcagaacggtgatagctatgggtatatgttgattccgaatgtagatgaaaacttgtatttccagcatcatcatcatcatcactaagctgagc

SEQ ID NO: 24 - protein, 653 aa, Streptococcus pneumoniae
MGSAWSHPQFEKASVKDTYTDRLDDWNGIIAGNQYYDSKNDQMAKLNQELEGKVADSLSSISSQADRIYLWEKFSNYKTSANLTATYRKLEEMAKQVTNPSSRYYQDETVVRTVRDSMEWMHKHVYNSEKSIVGNWWDYEIGTPRAINNTLSLMKEYFSDEEIKKYTDVIEKFVPDPEHFRKTTDNPFKALGGNLVDMGRVKVIAGLLRKDDQEISSTIRSIEQVFKLVDQGEGFYQDGSYIDHTNVAYTGAYGNVLIDGLSQLLPVIQKTKNPIDKDKMQTMYHWIDKSFAPLLVNGELMDMSRGRSISRANSEGHVAAVEVLRGIHRIADMSEGETKQRLQSLVKTIVQSDSYYDVFKNLKTYKDISLMQSLLSDAGVASVPRTSYLSAFNKMDKTAMYNAEKGFGFGLSLFSSRTLNYEHMNKENKRGWYTSDGMFYLYNGDLSHYSDGYWPTVNPYKMPGTTETDAKRADSDTGKVLPSAFVGTSKLDDANATATMDFTNWNQTLTAHKSWFMLKDKIAFLGSNIQNTSTDTAATTIDQRKLESSNPYKVYVNDKEASLTEQEKDYPETQSGFLESSDSKKNIGYFFFKKSSISMSKALQKGAWKDINEGQSDKEVENEFLTISQAHKQNGDSYGYMLIPNVDENLYFQ

SEQ ID NO: 25 - DNA, 1650 bp, Streptococcus pneumoniae
  source 1..1650: mol_type = genomic DNA
ccatgggcagcgcgtggagccatccgcagtttgaaaaagcaagcgtgaaggatacgtacacggaccgcttagatgactggaacgggatcatcgcggggaaccagtactatgatagcaaaaacgaccaaatggcaaaattaaatcaagaattagaaggtaaggtcgcggattcactctcaagcattagcagccaagctgaccggatctatttatgggaaaaatttagcaactataaaacaagcgccaaccttacggcaacgtatcgaaaattagaggagatggcaaagcaggtaacgaacccgagcagccgctattatcaggatgaaacggtcgtgcggacggtccgagattcaatggaatggatgcataaacatgtctacaacagcgaaaagtcaattgtggggaattggtgggattatgaaatcggcacgccgcgcgcaatcaataacacgttaagccttatgaaagaatacttcagcgatgaggaaattaaaaaatatacggatgtaattgaaaaatttgtcccagatccagaacatttccggaagacaacggataatccgttcaaggcgctcggcggtaatttagtggatatgggtcgagtcaaagtcatagcgggcttacttcgcaaggatgatcaggaaattagcagcacgattcggtcaattgagcaggtattcaagttagtcgaccagggcgaaggcttttatcaggatggttcatatatcgaccacacgaacgtggcatatacaggcgcgtatgggaacgtgttaattgatgggttgagccagcttttaccggtcattcagaagacgaagaacccgatcgataaagataaaatgcagacgatgtaccactggattgataaatcatttgcgccgttacttgtaaacggtgagcttatggatatgagccgcggtcggtcaatcagccgcgccaactcagaggggcacgtagcagccgtcgaagtcctccgggggattcaccggatagctgatatgagcgaaggtgaaacgaaacagcgcttacaaagcttagtaaagacgattgtgcagtcagatagctattatgatgtctttaagaacttaaagacgtataaggatatcagcttaatgcagtcattattaagcgatgccggtgtcgccagcgtgccgcggacgagctacctcagcgcatttaataagatggataaaacggcaatgtacaacgccgagaaagggtttggttttgggttaagcttgttttcaagccgcacgttaaactacgaacacatgaataaggaaaacaaacgcggctggtatacaagcgatgggatgttctatttatacaacggggatttaagccactattcagatgggtactggccgacggtgaacccgtataagatgccgggcacgacggagacagatgcgaagcgggctgattcagatacgggcaaagtgttacccagcgcgttcgtgggtacatcaaaactcgatgatgcaaacgctacggccacgatggatttcacgaattggaaccagacgttaacggcgcataagtcatggtttatgctcaaggataagattgcatttttaggttcaaacatccagaatacgagcacggaaaacttgtatttccagcatcatcatcatcatcactaagctgagc

SEQ ID NO: 26 - protein, 540 aa, Streptococcus pneumoniae
MGSAWSHPQFEKASVKDTYTDRLDDWNGIIAGNQYYDSKNDQMAKLNQELEGKVADSLSSISSQADRIYLWEKFSNYKTSANLTATYRKLEEMAKQVTNPSSRYYQDETVVRTVRDSMEWMHKHVYNSEKSIVGNWWDYEIGTPRAINNTLSLMKEYFSDEEIKKYTDVIEKFVPDPEHFRKTTDNPFKALGGNLVDMGRVKVIAGLLRKDDQEISSTIRSIEQVFKLVDQGEGFYQDGSYIDHTNVAYTGAYGNVLIDGLSQLLPVIQKTKNPIDKDKMQTMYHWIDKSFAPLLVNGELMDMSRGRSISRANSEGHVAAVEVLRGIHRIADMSEGETKQRLQSLVKTIVQSDSYYDVFKNLKTYKDISLMQSLLSDAGVASVPRTSYLSAFNKMDKTAMYNAEKGFGFGLSLFSSRTLNYEHMNKENKRGWYTSDGMFYLYNGDLSHYSDGYWPTVNPYKMPGTTETDAKRADSDTGKVLPSAFVGTSKLDDANATATMDFTNWNQTLTAHKSWFMLKDKIAFLGSNIQNTSTENLYFQ

SEQ ID NO: 27 - DNA, 1422 bp, Streptococcus pneumoniae
  source 1..1422: mol_type = genomic DNA
ccatgggcagcgcgtggagccatccgcagtttgaaaaagcaagcgtgaaggatacgtacacggaccgcttagatgactggaacgggatcatcgcggggaaccagtactatgatagcaaaaacgaccaaatggcaaaattaaatcaagaattagaaggtaaggtcgcggattcactctcaagcattagcagccaagctgaccggatctatttatgggaaaaatttagcaactataaaacaagcgccaaccttacggcaacgtatcgaaaattagaggagatggcaaagcaggtaacgaacccgagcagccgctattatcaggatgaaacggtcgtgcggacggtccgagattcaatggaatggatgcataaacatgtctacaacagcgaaaagtcaattgtggggaattggtgggattatgaaatcggcacgccgcgcgcaatcaataacacgttaagccttatgaaagaatacttcagcgatgaggaaattaaaaaatatacggatgtaattgaaaaatttgtcccagatccagaacatttccggaagacaacggataatccgttcaaggcgctcggcggtaatttagtggatatgggtcgagtcaaagtcatagcgggcttacttcgcaaggatgatcaggaaattagcagcacgattcggtcaattgagcaggtattcaagttagtcgaccagggcgaaggcttttatcaggatggttcatatatcgaccacacgaacgtggcatatacaggcgcgtatgggaacgtgttaattgatgggttgagccagcttttaccggtcattcagaagacgaagaacccgatcgataaagataaaatgcagacgatgtaccactggattgataaatcatttgcgccgttacttgtaaacggtgagcttatggatatgagccgcggtcggtcaatcagccgcgccaactcagaggggcacgtagcagccgtcgaagtcctccgggggattcaccggatagctgatatgagcgaaggtgaaacgaaacagcgcttacaaagcttagtaaagacgattgtgcagtcagatagctattatgatgtctttaagaacttaaagacgtataaggatatcagcttaatgcagtcattattaagcgatgccggtgtcgccagcgtgccgcggacgagctacctcagcgcatttaataagatggataaaacggcaatgtacaacgccgagaaagggtttggttttgggttaagcttgttttcaagccgcacgttaaactacgaacacatgaataaggaaaacaaacgcggctggtatacaagcgatgggatgttctatttatacaacggggatttaagccactattcagatgggtactggccgacggtgaacgaaaacttgtatttccagcatcatcatcatcatcactaagctgagc

SEQ ID NO: 28 - protein, 464 aa, Streptococcus pneumoniae
MGSAWSHPQFEKASVKDTYTDRLDDWNGIIAGNQYYDSKNDQMAKLNQELEGKVADSLSSISSQADRIYLWEKFSNYKTSANLTATYRKLEEMAKQVTNPSSRYYQDETVVRTVRDSMEWMHKHVYNSEKSIVGNWWDYEIGTPRAINNTLSLMKEYFSDEEIKKYTDVIEKFVPDPEHFRKTTDNPFKALGGNLVDMGRVKVIAGLLRKDDQEISSTIRSIEQVFKLVDQGEGFYQDGSYIDHTNVAYTGAYGNVLIDGLSQLLPVIQKTKNPIDKDKMQTMYHWIDKSFAPLLVNGELMDMSRGRSISRANSEGHVAAVEVLRGIHRIADMSEGETKQRLQSLVKTIVQSDSYYDVFKNLKTYKDISLMQSLLSDAGVASVPRTSYLSAFNKMDKTAMYNAEKGFGFGLSLFSSRTLNYEHMNKENKRGWYTSDGMFYLYNGDLSHYSDGYWPTVNENLYFQ

SEQ ID NO: 29 - DNA, 2271 bp, Streptococcus pneumoniae
  source 1..2271: mol_type = genomic DNA
ccatgggccatcatcatcatcatcacgatgatgatgataaaagcgcgtggagccatccgcagtttgaaaaagaaaacttgtatttccaggcaagcgtgaaggatacgtacacggaccgcttagatgactggaacgggatcatcgcggggaaccagtactatgatagcaaaaacgaccaaatggcaaaattaaatcaagaattagaaggtaaggtcgcggattcactctcaagcattagcagccaagctgaccggatctatttatgggaaaaatttagcaactataaaacaagcgccaaccttacggcaacgtatcgaaaattagaggagatggcaaagcaggtaacgaacccgagcagccgctattatcaggatgaaacggtcgtgcggacggtccgagattcaatggaatggatgcataaacatgtctacaacagcgaaaagtcaattgtggggaattggtgggattatgaaatcggcacgccgcgcgcaatcaataacacgttaagccttatgaaagaatacttcagcgatgaggaaattaaaaaatatacggatgtaattgaaaaatttgtcccagatccagaacatttccggaagacaacggataatccgttcaaggcgctcggcggtaatttagtggatatgggtcgagtcaaagtcatagcgggcttacttcgcaaggatgatcaggaaattagcagcacgattcggtcaattgagcaggtattcaagttagtcgaccagggcgaaggcttttatcaggatggttcatatatcgaccacacgaacgtggcatatacaggcgcgtatgggaacgtgttaattgatgggttgagccagcttttaccggtcattcagaagacgaagaacccgatcgataaagataaaatgcagacgatgtaccactggattgataaatcatttgcgccgttacttgtaaacggtgagcttatggatatgagccgcggtcggtcaatcagccgcgccaactcagaggggcacgtagcagccgtcgaagtcctccgggggattcaccggatagctgatatgagcgaaggtgaaacgaaacagcgcttacaaagcttagtaaagacgattgtgcagtcagatagctattatgatgtctttaagaacttaaagacgtataaggatatcagcttaatgcagtcattattaagcgatgccggtgtcgccagcgtgccgcggacgagctacctcagcgcatttaataagatggataaaacggcaatgtacaacgccgagaaagggtttggttttgggttaagcttgttttcaagccgcacgttaaactacgaacacatgaataaggaaaacaaacgcggctggtatacaagcgatgggatgttctatttatacaacggggatttaagccactattcagatgggtactggccgacggtgaacccgtataagatgccgggcacgacggagacagatgcgaagcgggctgattcagatacgggcaaagtgttacccagcgcgttcgtgggtacatcaaaactcgatgatgcaaacgctacggccacgatggatttcacgaattggaaccagacgttaacggcgcataagtcatggtttatgctcaaggataagattgcatttttaggttcaaacatccagaatacgagcacggatacggcggccacgacgattgaccaacggaaacttgaaagcagcaacccgtataaagtctatgtcaacgataaagaagcatcattaacggaacaggaaaaggattatccggaaacgcagagcgggtttttagaatcatcagattcaaaaaagaacattggctactttttctttaagaagagcagcatcagcatgagcaaggcgttacagaagggtgcatggaaggatatcaacgaaggtcaaagcgacaaggaagtggaaaacgaatttttaacaattagccaagcgcataagcagaacggtgatagctatgggtatatgttgattccgaatgtagatcgcgcaacgttcaaccagatgataaaagagttagaatcatcattgatcgaaaacaatgaaacgttacaaagcgtgtatgatgcaaaacagggtgtgtgggggattgtaaaatatgatgatagcgtcagcacgatttcaaatcagttccaggtgttaaaacgcggtgtctatacgattcggaaagaaggggatgaatataagattgcatactataacccggaaacgcaagaaagcgcgccggatcaagaagtctttaaaaagctcgagtaagctgagc

SEQ ID NO: 30 - protein, 724 aa, Streptococcus pneumoniae; identical to SEQ ID NO: 9

SEQ ID NO: 31 - DNA, 2043 bp, Streptococcus pneumoniae
  source 1..2043: mol_type = genomic DNA
ccatgggccatcatcatcatcatcacgatgatgatgataaaagcgcgtggagccatccgcagtttgaaaaagaaaacttgtatttccaggcaagcgtgaaggatacgtacacggaccgcttagatgactggaacgggatcatcgcggggaaccagtactatgatagcaaaaacgaccaaatggcaaaattaaatcaagaattagaaggtaaggtcgcggattcactctcaagcattagcagccaagctgaccggatctatttatgggaaaaatttagcaactataaaacaagcgccaaccttacggcaacgtatcgaaaattagaggagatggcaaagcaggtaacgaacccgagcagccgctattatcaggatgaaacggtcgtgcggacggtccgagattcaatggaatggatgcataaacatgtctacaacagcgaaaagtcaattgtggggaattggtgggattatgaaatcggcacgccgcgcgcaatcaataacacgttaagccttatgaaagaatacttcagcgatgaggaaattaaaaaatatacggatgtaattgaaaaatttgtcccagatccagaacatttccggaagacaacggataatccgttcaaggcgctcggcggtaatttagtggatatgggtcgagtcaaagtcatagcgggcttacttcgcaaggatgatcaggaaattagcagcacgattcggtcaattgagcaggtattcaagttagtcgaccagggcgaaggcttttatcaggatggttcatatatcgaccacacgaacgtggcatatacaggcgcgtatgggaacgtgttaattgatgggttgagccagcttttaccggtcattcagaagacgaagaacccgatcgataaagataaaatgcagacgatgtaccactggattgataaatcatttgcgccgttacttgtaaacggtgagcttatggatatgagccgcggtcggtcaatcagccgcgccaactcagaggggcacgtagcagccgtcgaagtcctccgggggattcaccggatagctgatatgagcgaaggtgaaacgaaacagcgcttacaaagcttagtaaagacgattgtgcagtcagatagctattatgatgtctttaagaacttaaagacgtataaggatatcagcttaatgcagtcattattaagcgatgccggtgtcgccagcgtgccgcggacgagctacctcagcgcatttaataagatggataaaacggcaatgtacaacgccgagaaagggtttggttttgggttaagcttgttttcaagccgcacgttaaactacgaacacatgaataaggaaaacaaacgcggctggtatacaagcgatgggatgttctatttatacaacggggatttaagccactattcagatgggtactggccgacggtgaacccgtataagatgccgggcacgacggagacagatgcgaagcgggctgattcagatacgggcaaagtgttacccagcgcgttcgtgggtacatcaaaactcgatgatgcaaacgctacggccacgatggatttcacgaattggaaccagacgttaacggcgcataagtcatggtttatgctcaaggataagattgcatttttaggttcaaacatccagaatacgagcacggatacggcggccacgacgattgaccaacggaaacttgaaagcagcaacccgtataaagtctatgtcaacgataaagaagcatcattaacggaacaggaaaaggattatccggaaacgcagagcgggtttttagaatcatcagattcaaaaaagaacattggctactttttctttaagaagagcagcatcagcatgagcaaggcgttacagaagggtgcatggaaggatatcaacgaaggtcaaagcgacaaggaagtggaaaacgaatttttaacaattagccaagcgcataagcagaacggtgatagctatgggtatatgttgattccgaatgtagatcgcgcaacgttcaaccagatgataaaagagttagaatcataagctgagc

SEQ ID NO: 32 - protein, 648 aa, Streptococcus pneumoniae
ASVKDTYTDRLDDWNGIIAGNQYYDSKNDQMAKLNQELEGKVADSLSSISSQADRIYLWEKFSNYKTSANLTATYRKLEEMAKQVTNPSSRYYQDETVVRTVRDSMEWMHKHVYNSEKSIVGNWWDYEIGTPRAINNTLSLMKEYFSDEEIKKYTDVIEKFVPDPEHFRKTTDNPFKALGGNLVDMGRVKVIAGLLRKDDQEISSTIRSIEQVFKLVDQGEGFYQDGSYIDHTNVAYTGAYGNVLIDGLSQLLPVIQKTKNPIDKDKMQTMYHWIDKSFAPLLVNGELMDMSRGRSISRANSEGHVAAVEVLRGIHRIADMSEGETKQRLQSLVKTIVQSDSYYDVFKNLKTYKDISLMQSLLSDAGVASVPRTSYLSAFNKMDKTAMYNAEKGFGFGLSLFSSRTLNYEHMNKENKRGWYTSDGMFYLYNGDLSHYSDGYWPTVNPYKMPGTTETDAKRADSDTGKVLPSAFVGTSKLDDANATATMDFTNWNQTLTAHKSWFMLKDKIAFLGSNIQNTSTDTAATTIDQRKLESSNPYKVYVNDKEASLTEQEKDYPETQSGFLESSDSKKNIGYFFFKKSSISMSKALQKGAWKDINEGQSDKEVENEFLTISQAHKQNGDSYGYMLIPNVDRATFNQMIKELES

SEQ ID NO: 33 - DNA, 2004 bp, Streptococcus pneumoniae
  source 1..2004: mol_type = genomic DNA
ccatgggccatcatcatcatcatcacgatgatgatgataaaagcgcgtggagccatccgcagtttgaaaaagaaaacttgtatttccaggcaagcgtgaaggatacgtacacggaccgcttagatgactggaacgggatcatcgcggggaaccagtactatgatagcaaaaacgaccaaatggcaaaattaaatcaagaattagaaggtaaggtcgcggattcactctcaagcattagcagccaagctgaccggatctatttatgggaaaaatttagcaactataaaacaagcgccaaccttacggcaacgtatcgaaaattagaggagatggcaaagcaggtaacgaacccgagcagccgctattatcaggatgaaacggtcgtgcggacggtccgagattcaatggaatggatgcataaacatgtctacaacagcgaaaagtcaattgtggggaattggtgggattatgaaatcggcacgccgcgcgcaatcaataacacgttaagccttatgaaagaatacttcagcgatgaggaaattaaaaaatatacggatgtaattgaaaaatttgtcccagatccagaacatttccggaagacaacggataatccgttcaaggcgctcggcggtaatttagtggatatgggtcgagtcaaagtcatagcgggcttacttcgcaaggatgatcaggaaattagcagcacgattcggtcaattgagcaggtattcaagttagtcgaccagggcgaaggcttttatcaggatggttcatatatcgaccacacgaacgtggcatatacaggcgcgtatgggaacgtgttaattgatgggttgagccagcttttaccggtcattcagaagacgaagaacccgatcgataaagataaaatgcagacgatgtaccactggattgataaatcatttgcgccgttacttgtaaacggtgagcttatggatatgagccgcggtcggtcaatcagccgcgccaactcagaggggcacgtagcagccgtcgaagtcctccgggggattcaccggatagctgatatgagcgaaggtgaaacgaaacagcgcttacaaagcttagtaaagacgattgtgcagtcagatagctattatgatgtctttaagaacttaaagacgtataaggatatcagcttaatgcagtcattattaagcgatgccggtgtcgccagcgtgccgcggacgagctacctcagcgcatttaataagatggataaaacggcaatgtacaacgccgagaaagggtttggttttgggttaagcttgttttcaagccgcacgttaaactacgaacacatgaataaggaaaacaaacgcggctggtatacaagcgatgggatgttctatttatacaacggggatttaagccactattcagatgggtactggccgacggtgaacccgtataagatgccgggcacgacggagacagatgcgaagcgggctgattcagatacgggcaaagtgttacccagcgcgttcgtgggtacatcaaaactcgatgatgcaaacgctacggccacgatggatttcacgaattggaaccagacgttaacggcgcataagtcatggtttatgctcaaggataagattgcatttttaggttcaaacatccagaatacgagcacggatacggcggccacgacgattgaccaacggaaacttgaaagcagcaacccgtataaagtctatgtcaacgataaagaagcatcattaacggaacaggaaaaggattatccggaaacgcagagcgggtttttagaatcatcagattcaaaaaagaacattggctactttttctttaagaagagcagcatcagcatgagcaaggcgttacagaagggtgcatggaaggatatcaacgaaggtcaaagcgacaaggaagtggaaaacgaatttttaacaattagccaagcgcataagcagaacggtgatagctatgggtatatgttgattccgaatgtagattaagctgagc

SEQ ID NO: 34 - protein, 635 aa, Streptococcus pneumoniae
ASVKDTYTDRLDDWNGIIAGNQYYDSKNDQMAKLNQELEGKVADSLSSISSQADRIYLWEKFSNYKTSANLTATYRKLEEMAKQVTNPSSRYYQDETVVRTVRDSMEWMHKHVYNSEKSIVGNWWDYEIGTPRAINNTLSLMKEYFSDEEIKKYTDVIEKFVPDPEHFRKTTDNPFKALGGNLVDMGRVKVIAGLLRKDDQEISSTIRSIEQVFKLVDQGEGFYQDGSYIDHTNVAYTGAYGNVLIDGLSQLLPVIQKTKNPIDKDKMQTMYHWIDKSFAPLLVNGELMDMSRGRSISRANSEGHVAAVEVLRGIHRIADMSEGETKQRLQSLVKTIVQSDSYYDVFKNLKTYKDISLMQSLLSDAGVASVPRTSYLSAFNKMDKTAMYNAEKGFGFGLSLFSSRTLNYEHMNKENKRGWYTSDGMFYLYNGDLSHYSDGYWPTVNPYKMPGTTETDAKRADSDTGKVLPSAFVGTSKLDDANATATMDFTNWNQTLTAHKSWFMLKDKIAFLGSNIQNTSTDTAATTIDQRKLESSNPYKVYVNDKEASLTEQEKDYPETQSGFLESSDSKKNIGYFFFKKSSISMSKALQKGAWKDINEGQSDKEVENEFLTISQAHKQNGDSYGYMLIPNVD

SEQ ID NO: 35 - DNA, 1665 bp, Streptococcus pneumoniae
  source 1..1665: mol_type = genomic DNA
ccatgggccatcatcatcatcatcacgatgatgatgataaaagcgcgtggagccatccgcagtttgaaaaagaaaacttgtatttccaggcaagcgtgaaggatacgtacacggaccgcttagatgactggaacgggatcatcgcggggaaccagtactatgatagcaaaaacgaccaaatggcaaaattaaatcaagaattagaaggtaaggtcgcggattcactctcaagcattagcagccaagctgaccggatctatttatgggaaaaatttagcaactataaaacaagcgccaaccttacggcaacgtatcgaaaattagaggagatggcaaagcaggtaacgaacccgagcagccgctattatcaggatgaaacggtcgtgcggacggtccgagattcaatggaatggatgcataaacatgtctacaacagcgaaaagtcaattgtggggaattggtgggattatgaaatcggcacgccgcgcgcaatcaataacacgttaagccttatgaaagaatacttcagcgatgaggaaattaaaaaatatacggatgtaattgaaaaatttgtcccagatccagaacatttccggaagacaacggataatccgttcaaggcgctcggcggtaatttagtggatatgggtcgagtcaaagtcatagcgggcttacttcgcaaggatgatcaggaaattagcagcacgattcggtcaattgagcaggtattcaagttagtcgaccagggcgaaggcttttatcaggatggttcatatatcgaccacacgaacgtggcatatacaggcgcgtatgggaacgtgttaattgatgggttgagccagcttttaccggtcattcagaagacgaagaacccgatcgataaagataaaatgcagacgatgtaccactggattgataaatcatttgcgccgttacttgtaaacggtgagcttatggatatgagccgcggtcggtcaatcagccgcgccaactcagaggggcacgtagcagccgtcgaagtcctccgggggattcaccggatagctgatatgagcgaaggtgaaacgaaacagcgcttacaaagcttagtaaagacgattgtgcagtcagatagctattatgatgtctttaagaacttaaagacgtataaggatatcagcttaatgcagtcattattaagcgatgccggtgtcgccagcgtgccgcggacgagctacctcagcgcatttaataagatggataaaacggcaatgtacaacgccgagaaagggtttggttttgggttaagcttgttttcaagccgcacgttaaactacgaacacatgaataaggaaaacaaacgcggctggtatacaagcgatgggatgttctatttatacaacggggatttaagccactattcagatgggtactggccgacggtgaacccgtataagatgccgggcacgacggagacagatgcgaagcgggctgattcagatacgggcaaagtgttacccagcgcgttcgtgggtacatcaaaactcgatgatgcaaacgctacggccacgatggatttcacgaattggaaccagacgttaacggcgcataagtcatggtttatgctcaaggataagattgcatttttaggttcaaacatccagaatacgagcacgtaagctgagc

SEQ ID NO: 36 - protein, 522 aa, Streptococcus pneumoniae
ASVKDTYTDRLDDWNGIIAGNQYYDSKNDQMAKLNQELEGKVADSLSSISSQADRIYLWEKFSNYKTSANLTATYRKLEEMAKQVTNPSSRYYQDETVVRTVRDSMEWMHKHVYNSEKSIVGNWWDYEIGTPRAINNTLSLMKEYFSDEEIKKYTDVIEKFVPDPEHFRKTTDNPFKALGGNLVDMGRVKVIAGLLRKDDQEISSTIRSIEQVFKLVDQGEGFYQDGSYIDHTNVAYTGAYGNVLIDGLSQLLPVIQKTKNPIDKDKMQTMYHWIDKSFAPLLVNGELMDMSRGRSISRANSEGHVAAVEVLRGIHRIADMSEGETKQRLQSLVKTIVQSDSYYDVFKNLKTYKDISLMQSLLSDAGVASVPRTSYLSAFNKMDKTAMYNAEKGFGFGLSLFSSRTLNYEHMNKENKRGWYTSDGMFYLYNGDLSHYSDGYWPTVNPYKMPGTTETDAKRADSDTGKVLPSAFVGTSKLDDANATATMDFTNWNQTLTAHKSWFMLKDKIAFLGSNIQNTST

SEQ ID NO: 37 - DNA, 1437 bp, Streptococcus pneumoniae
  source 1..1437: mol_type = genomic DNA
ccatgggccatcatcatcatcatcacgatgatgatgataaaagcgcgtggagccatccgcagtttgaaaaagaaaacttgtatttccaggcaagcgtgaaggatacgtacacggaccgcttagatgactggaacgggatcatcgcggggaaccagtactatgatagcaaaaacgaccaaatggcaaaattaaatcaagaattagaaggtaaggtcgcggattcactctcaagcattagcagccaagctgaccggatctatttatgggaaaaatttagcaactataaaacaagcgccaaccttacggcaacgtatcgaaaattagaggagatggcaaagcaggtaacgaacccgagcagccgctattatcaggatgaaacggtcgtgcggacggtccgagattcaatggaatggatgcataaacatgtctacaacagcgaaaagtcaattgtggggaattggtgggattatgaaatcggcacgccgcgcgcaatcaataacacgttaagccttatgaaagaatacttcagcgatgaggaaattaaaaaatatacggatgtaattgaaaaatttgtcccagatccagaacatttccggaagacaacggataatccgttcaaggcgctcggcggtaatttagtggatatgggtcgagtcaaagtcatagcgggcttacttcgcaaggatgatcaggaaattagcagcacgattcggtcaattgagcaggtattcaagttagtcgaccagggcgaaggcttttatcaggatggttcatatatcgaccacacgaacgtggcatatacaggcgcgtatgggaacgtgttaattgatgggttgagccagcttttaccggtcattcagaagacgaagaacccgatcgataaagataaaatgcagacgatgtaccactggattgataaatcatttgcgccgttacttgtaaacggtgagcttatggatatgagccgcggtcggtcaatcagccgcgccaactcagaggggcacgtagcagccgtcgaagtcctccgggggattcaccggatagctgatatgagcgaaggtgaaacgaaacagcgcttacaaagcttagtaaagacgattgtgcagtcagatagctattatgatgtctttaagaacttaaagacgtataaggatatcagcttaatgcagtcattattaagcgatgccggtgtcgccagcgtgccgcggacgagctacctcagcgcatttaataagatggataaaacggcaatgtacaacgccgagaaagggtttggttttgggttaagcttgttttcaagccgcacgttaaactacgaacacatgaataaggaaaacaaacgcggctggtatacaagcgatgggatgttctatttatacaacggggatttaagccactattcagatgggtactggccgacggtgaactaagctgagc

SEQ ID NO: 38 - protein, 446 aa, Streptococcus pneumoniae
ASVKDTYTDRLDDWNGIIAGNQYYDSKNDQMAKLNQELEGKVADSLSSISSQADRIYLWEKFSNYKTSANLTATYRKLEEMAKQVTNPSSRYYQDETVVRTVRDSMEWMHKHVYNSEKSIVGNWWDYEIGTPRAINNTLSLMKEYFSDEEIKKYTDVIEKFVPDPEHFRKTTDNPFKALGGNLVDMGRVKVIAGLLRKDDQEISSTIRSIEQVFKLVDQGEGFYQDGSYIDHTNVAYTGAYGNVLIDGLSQLLPVIQKTKNPIDKDKMQTMYHWIDKSFAPLLVNGELMDMSRGRSISRANSEGHVAAVEVLRGIHRIADMSEGETKQRLQSLVKTIVQSDSYYDVFKNLKTYKDISLMQSLLSDAGVASVPRTSYLSAFNKMDKTAMYNAEKGFGFGLSLFSSRTLNYEHMNKENKRGWYTSDGMFYLYNGDLSHYSDGYWPTVN

(19) United States
(12) Patent Application Publication
(10) Pub. No.: US 2024/0417710 A1
(43) Pub. Date: Dec. 19, 2024

(54) A PRODUCTION OF A PURIFIED MODIFIED BACTERIAL HYALURONIDASE POLYPEPTIDE, PHARMACEUTICAL COMPOSITIONS AND THEIR USES

(71) Applicant: PHARMACT HOLDING AG, Baar (CH)

(72) Inventors: Gunther BURGARD, Homburg (DE); Rainer BOEHNKE, Berlin (DE); Sven BENSON, Stuttgart (DE); Lenz LORENZ, Sindelfingen (DE); Philipp SCHELLENBERGER, Remshalden (DE)

(21) Appl. No.: 18/703,314

(22) PCT Filed: Oct. 19, 2022
(86) PCT No.: PCT/EP2022/079111

§ 371 Date: Apr. 19, 2024

(30) Foreign Application Priority Data
Oct. 19, 2021 (DE) 10 2021 127 066.9

(51) Int. Cl. C12N 9/26; A61K 38/47; C12N 15/70

(52) U.S. Cl. CPC C12N 9/2474; A61K 38/47; C12N 15/70; C07K 2319/21; C07K 2319/22

(57) ABSTRACT

Disclosed is a production process of a purified modified bacterial hyaluronidase polypeptide, a pharmaceutical composition including the inventively obtainable purified modified bacterial hyaluronidase polypeptide and its uses.

### TECHNICAL FIELD

The present invention relates to a production process of a purified modified bacterial hyaluronidase polypeptide, a pharmaceutical composition comprising the inventively obtainable purified modified bacterial hyaluronidase polypeptide and its uses.

### PRIOR ART

Hyaluronic acid is an essential component of the extracellular matrix and a quantitatively significant constituent of the interstitial barrier. Hyaluronidase is a hydrolytic enzyme that cleaves hyaluronic acid in D-glucuronic acid and N-acetyl glucosamine, increasing the permeability of the interstitial matrix. Hyaluronidase is widely distributed in nature. In the human, six different hyaluronidases, HYAL1-4, HYAL-P1 and PH-20, have been identified, wherein PH-20 is regarded to exert the strongest biologic activity.

Today, animal-derived bovine or ovine testicular hyaluronidases as well as synthetic hyaluronidases are clinically applied as adjuncts to increase the bioavailability of drugs, for the therapy of extravasations, or for the management of complications associated with the aesthetic injection of hyaluronic acid-based fillers.

While hyaluronidase derived from animal origin imparts a risk of transmitting animal diseases, such spongiform encephalopathy, human and bacterial recombinant hyaluronidase exhibit a higher purity, which reduces pharmaceutical risks.

In order to meet the clinical need, it is an aim of the present invention to provide a hyaluronidase polypeptide that is suitable for pharmaceutical application and in particular exhibits a suitable high purity degree, and/or a suitable specific activity, suitable stability and solubility and at the same time exhibits a time and cost effective production process.

### BRIEF DESCRIPTION OF THE INVENTION

The aforementioned aim is solved at least in part by means of the claimed inventive subject matter. Advantages (preferred embodiments) are set out in the detailed description hereinafter and/or the accompanying figures as well as in the dependent claims.

Accordingly, a first aspect of the invention relates to a process of production of a purified bacterial hyaluronidase polypeptide comprising or consisting of the following steps:
a. Culturing a transformed host cell with recombinant expression vector comprising or consisting of a nucleic acid encoding a modified bacterial hyaluronidase polypeptide in a suitable growth medium under suitable growth conditions to express the bacterial hyaluronidase polypeptide comprising or consisting of at least 90% sequence identity to SEQ ID No. 1, wherein the hyaluronidase polypeptide comprises a C-terminal tag and an N-terminal tag,
b. Harvesting the cultured transformed host cell of step a),
c. Lysing the harvested host cells of step b) and separating resulting host cell fragments from resulting host cell content comprising the bacterial hyaluronidase polypeptide, and
d. Purifying the resulting host cell content of step c) with a first affinity chromatography corresponding to the C-terminal tag and a second affinity chromatography corresponding to the N-terminal tag to result in a purified form of the bacterial hyaluronidase polypeptide comprising or consisting of at least 90% sequence identity to SEQ ID No. 1, wherein the hyaluronidase polypeptide comprises the C-terminal tag and the N-terminal tag, and
e. Removing at least part of the C-terminal tag of the purified bacterial hyaluronidase of step d) and/or removing at least part of the N-terminal tag of the purified bacterial hyaluronidase of step d).

According to an alternative of the first aspect, the invention relates to a process of production of a purified modified bacterial hyaluronidase polypeptide comprising or consisting of at least 90% sequence identity of any one of SEQ ID Nos. 9, 10, 20, 22, 24, 26 or 28 comprising or consisting of the following steps:
a. Culturing a transformed host cell with recombinant expression vector comprising or consisting of a nucleic acid having at least 90% identity to any one of SEQ ID Nos: 19, 21, 23, 25, or 27 encoding a modified bacterial hyaluronidase polypeptide in a suitable growth medium under suitable growth conditions to express the bacterial hyaluronidase polypeptide, wherein the hyaluronidase polypeptide comprises a C-terminal tag and an N-terminal tag,
b. Harvesting the cultured transformed host cell of step a),
c. Lysing the harvested host cells of step b) and separating resulting host cell fragments from resulting host cell content comprising the bacterial hyaluronidase polypeptide, and
d. Purifying the resulting host cell content of step c) with a first affinity chromatography corresponding to the C-terminal tag and a second affinity chromatography corresponding to the N-terminal tag to result in a purified form of the bacterial hyaluronidase polypeptide, and
e. Removing at least part of the C-terminal tag of the purified bacterial hyaluronidase of step d) and/or removing at least part of the N-terminal tag of the purified bacterial hyaluronidase of step d) in order to result in the modified bacterial hyaluronidase comprising or consisting of at least 90% sequence identity of SEQ ID Nos. 9, 10, 20, 22, 24, 26 or 28.

A second aspect of the present invention relates to a modified bacterial hyaluronidase polypeptide comprising or consisting of at least 90% sequence identity to SEQ ID No. 9 or SEQ ID No. 10 obtainable according to the production process according to the first inventive aspect, wherein the inventively obtainable purified modified bacterial hyaluronidase polypeptide may respectively comprise no, one or two remaining residues of the C-terminal tag and/or no, one or two remaining residues of the N-terminal tag.

According to an alternative, the second inventive aspect relates to a modified bacterial hyaluronidase polypeptide consisting of 99.5%, 99.6, 99.7, 99.8, 99.9, 100% sequence identity to any one of SEQ ID Nos. 9, 10, 20, 22, 24, 26, 28, 30, 32, 34, 36, or 38.

A third aspect of the present invention relates to a pharmaceutical composition comprising the modified bacterial hyaluronidase polypeptide according the second inventive aspect in a therapeutically effective amount and one or more pharmaceutically acceptable excipients, wherein the pharmaceutical composition is used in the treatment or prophylaxis of a hyaluronan-associated and/or proteoglycan-associated disease or disorder, preferably selected from a group consisting homozygous familial hypercholesterolemia (xanthomatose), heterozygous familial hypercholesterolemia, diabetic foot syndrome, arterial hypertension or cardiac insufficiency.

A fourth aspect of the present invention relates to a method of treating a hyaluronan-associated and/or proteoglycan-associated disease or disorder comprising or consisting of administering the modified bacterial hyaluronidase polypeptide according to the second inventive aspect, or the pharmaceutical composition according to the third inventive aspect to a subject in need thereof, preferably wherein the hyaluronan-associated and/or proteoglycan-associated disease or disorder is selected from a group consisting of a homozygous familial hypercholesterolemia (xanthomatose), heterozygous familial hypercholesterolemia, diabetic foot syndrome, arterial hypertension or cardiac insufficiency.

A fifth aspect of the present invention relates to an alternative process of production of a purified modified bacterial hyaluronidase polypeptide of at least 90% sequence identity of any one of SEQ ID Nos. 9, 10, 30, 32, 34, 36, or 38 comprising or consisting of the following steps:
a. Culturing a transformed host cell with recombinant expression vector comprising or consisting of a nucleic acid of at least 90% sequence identity of any one of SEQ ID Nos. 29, 31, 33, 35, or 37 encoding a modified bacterial hyaluronidase polypeptide in a suitable growth medium under suitable growth conditions to express the bacterial hyaluronidase polypeptide, wherein the bacterial hyaluronidase polypeptide comprises a N-terminal tag including at least a first N-terminal purification tag connected by a cleavage site to a second different purification tag which is connected to the hyaluronidase polypeptide either directly or by a second cleavage site,
b. Harvesting the cultured transformed host cell of step a),
c. Lysing the harvested host cells of step b) and separating resulting host cell fragments from resulting host cell content comprising the bacterial hyaluronidase polypeptide, and
d. Purifying the resulting host cell content of step c) with a first affinity chromatography corresponding to the N-terminal first purification tag, removing the first purification tag from the remaining hyaluronidase sequence and purifying the resulting hyaluronidase sequence with a second affinity chromatography corresponding to the second purification tag and optionally removing the N-terminal second purification tag to result in a purified form of the bacterial hyaluronidase polypeptide comprising or consisting of at least 90% sequence identity to SEQ ID Nos. 9, 10, 30, 32, 34, 36, or 38.

The inventive aspects of the present invention as disclosed hereinbefore can comprise any possible (sub-) combination of the inventive aspects and preferred embodiments thereof as set out in the dependent claims or as disclosed in the following detailed description and/or in the accompanying figures, provided the resulting combination of features is reasonable to a person skilled in the art.

### BRIEF DESCRIPTION OF THE DRAWINGS

Further characteristics and advantages of the present invention will ensue from the accompanying drawings, wherein

FIG. 1 represents a Vector Map plasmid of an inventive recombinant expression vector.

FIG. 2 represents a scan of a stained gel after SDS-Page analysis comprising reference protein (bovine serum albumin, syn: BSA) bands of different concentrations, ladder protein bands and bands of inventive modified bacterial hyaluronidase of different concentrations.

FIGS. 3a) to c) represent images of LB-Agar Plates comprising E. coli positive control FIG. 3a), LB-plate negative control FIG. 3b) and inventive d016 sample FIG. 3c) over a time period of 4 days.

FIGS. 4a) and b) represent a schematic process for removing an N-terminal purification tag and a schematic process for removing one or two C-terminal purification tags.

### DETAILED DESCRIPTION OF THE INVENTION

As set out in more detail hereinafter, the inventors of the different aspects of the present invention have found out that the inventive production process produces a purified modified bacterial hyaluronidase polypeptide comprising or consisting of at least 90% sequence identity to SEQ ID Nos. 9, 10, 20, 22, 24, 26, 28, 30, 32, 34, 36, or 38, wherein at least part of the previously purification tags are removed, and wherein the inventive modified bacterial hyaluronidase polypeptide exhibits a high purity, in particular of >95% or >98.8% (see example section 1.4.1) and a high Specific Activity, in particular of 1,500,000 U/mg (see example section 1.4.2).

In contrast thereto, comparative hyaluronidases, such as bovine hyaluronidases exhibit a wide range of lower Specific Activities, namely in the range of 300 to 15,000 U/mg (see example section 2.3). PH20, regarded as the most active out of the human hyaluronidases, also exhibits a lower Specific Activity, namely in the range of 40,000 and 50,000 U/mg. The Specific Activity of bacterial hyaluronidase derived from Streptomyces koganeiensis is comparable to the Specific Activity of PH20 and, thus, is also lower than the Specific Activity of the inventive modified bacterial hyaluronidase.

Moreover, the inventive modified bacterial hyaluronidase exhibits suitable stability and solubility, which is shown in example section 1.4.5 below. The increased stability, including stability against (exo-) peptidases (half life) may be due to the use of a remaining respective C-terminal or N-terminal tag. The respective tags may also increase the solubility of the inventive modified bacterial hyaluronidase in comparison to the wild-type hyaluronidase (see SEQ ID No. 3, DNA encoding wild-type hyaluronidase see SEQ ID. No. 4). Due to the increased stability and solubility properties, the inventive modified bacterial hyaluronidase is preferred for formulating pharmaceutical compositions, in particular parenteral injection compositions. Alternatively, in case the C-terminal and/or N-terminal purification tag has been removed from the inventive modified bacterial hyaluronidase variant, the suitable solubility of the inventive modified bacterial hyaluronidase may be achieved with other suitable amino acid residues.

Furthermore, the inventive modified bacterial hyaluronidase sequences of SEQ ID Nos. 22, 24, 26, 28, 32, 34, 36, or 38 may furthermore be advantageous, as they are reduced in size in comparison to the hyaluronidase sequence of d016 of SEQ ID 1 and, thus, show an increased efficacy in small capillary vessels, or show an increased transmission between different physiological compartments, in particular when crossing the blood-brain barrier.

Thus, the inventive production process for providing a purified modified bacterial hyaluronidase provides a comparatively high yield and at the same time, a high purity and high Specific Activity already in laboratory scale and is in view of the production steps time and cost effective. Furthermore, the embodiments may be optimized with respect to the sequence size in order to increase the efficacy in small capillary vessels and/or increase the transmission of the blood-brain barrier.

In the context of the present invention, the expression “modified bacterial hyaluronidase polypeptide comprising or consisting of at least 90% sequence identity to SEQ ID No. 1” means that the modified bacterial hyaluronidase of SEQ ID No. 1 comprises at least 90, 91, 92, 93, 94, 95, 96, 97, 98, 99, 99.5, 99.6, 99.7, 99.8, 99.9%, of the sequence of SEQ ID No. 1 or that it consists of 100% of SEQ ID No. 1, wherein the SEQ ID No. 1 comprises a C-terminal tag, preferably a HIS tag of SEQ ID No. 7 and comprises an N-terminal tag, preferably a STREP tag of SEQ ID No. 5. In case that the sequence of the hyaluronidase consists of 100% of the SEQ ID No. 1, the inventive hyaluronidase is also synonymously called “d016tag”.

In the context of the present invention, the expression “inventive modified bacterial hyaluronidase polypeptide comprising or consisting of at least 90% sequence identity to SEQ ID No. 9 or SEQ ID No. 10” means that the inventive modified bacterial hyaluronidase of SEQ ID No. 9 or SEQ ID No. 10 respectively comprises at least 90, 91, 92, 93, 94, 95, 96, 97, 98, 99, 99.5, 99.6, 99.7, 99.8, 99.9%, of the sequence of SEQ ID No. 9 or 10 or that it consists of 100% of SEQ ID No. 9 or 10. The respective C-terminal tag, preferably the HIS tag of SEQ ID No. 7 and/or the N-terminal tag, preferably the STREP tag of SEQ ID No. 5, may at least in part be removed in the inventive modified bacterial hyaluronidase of SEQ ID No. 9 or SEQ ID No. 10. In other words, the inventive modified bacterial hyaluronidase of SEQ ID No. 9 or SEQ ID No. 10 may respectively comprise no, one or two remaining residues of a previously comprised C-terminal tag, preferably the HIS tag and/or no, one, two, three, or more residues of the N-terminal tag, preferably STREP tag. Thus, a sequence of the inventive hyaluronidase consists of SEQ ID No. 9 or SEQ ID No. 10 including no, one, two or three amino acid residues at the C-terminus, preferably wherein the amino acid residues are respectively histidine residues, and no, one, two, three, or more residues at the N-terminus, preferably wherein the one, two, three or more amino acid residues are preferably selected from the STREP tag of SEQ ID No. 5, and wherein such a inventively obtainable purified bacterial hyaluronidase is also synonymously called “d016”. The inventive modified bacterial hyaluronidase polypeptide is optionally suitably adapted for passing the blood-brain barrier and/or is simultaneously or sequentially applied with active ingredients increasing the blood-brain barrier efflux inhibitory agents.

According to an alternative, the inventive modified bacterial hyaluronidase of SEQ ID No. 9 may comprise at the N-terminal site a sequence residue of SEQ ID No. 11 and at the C-terminal site a sequence residue of SEQ ID No. 12.

Furthermore, the “inventive modified bacterial hyaluronidase polypeptide comprising or consisting of at least 90% sequence identity to SEQ ID Nos. 9, 10, 20, 22, 24, 26 or 28” or “inventive modified bacterial hyaluronidase polypeptide comprising or consisting of at least 90% sequence identity to SEQ ID Nos. 9, 10, 30, 32, 34, 36, or 38” respectively mean that the inventive modified bacterial hyaluronidase of SEQ ID Nos. 9, 10, 20, 22, 24, 26 or 28 or of SEQ ID Nos. 9, 10, 30, 32, 34, 36, or 38 respectively comprises at least 90, 91, 92, 93, 94, 95, 96, 97, 98, 99, 99.5, 99.6, 99.7, 99.8, 99.9%, sequence identity of the respective sequences of SEQ ID Nos. 9, 10, 20, 22, 24, 26, 28 30, 32, 34, 36, or 38 or that it consists of 100% of SEQ ID Nos. 9, 10, 20, 22, 24, 26, 28 30, 32, 34, 36, or 38.

In accordance with the present invention, sequence identity is calculated using an ungapped sequence alignment, for example using the BLAST alignment tool.

In comparison to the modified bacterial hyaluronidases having at least 90% sequence identity of SEQ ID No. 1 and comprising the C-terminal tag, preferably HIS tag, and the N-terminal tag, preferably STREP tag, the inventively obtainable purified modified bacterial hyaluronidases have a lower risk for immunogenicity due to the reduced tag residues. Preferably, at least the HIS tag is at least partly removed, more preferably, wherein only one or no amino acid residue of the HIS tag remain in the inventively obtainable hyaluronidase sequence. The STREP tag may remain in the inventively obtainable hyaluronidase or alternatively, no, one, two, three or four residues of the STREP tag remain in the inventively obtainable hyaluronidase sequence.

Removal of the respective or or more purification tags can be facilitated by encoding a cleaving site in the encoding DNA of the expression vector, such as a protease cleaving site, e.g. TEV cleavage sequence of SEQ ID No. 15, or an enterokinase cleavage sequence, e.g. enterokinase (light chain) cleavage site of SEQ ID No. 17 into the DNA sequence encoding the inventive hyaluronidase. The respective amino acid sequences of the respective cleavage sites relate to, e.g., TEV cleavage site of SEQ ID No. 16 and Enterokinase (light chain) cleavage site of SEQ ID No. 18.

Thus, the inventive modified bacterial hyaluronidase obtainable according to the inventive process according to the first and fifth inventive aspect is suitable for use in a pharmaceutical composition.

In view of the presented comparatively high Specific Activity, purity, stability, solubility and safety profiles, the inventively obtainable purified modified bacterial hyaluronidase, preferably d016 or the variants thereof according to the inventive aspects five and six are suitable for use in the treatment or prophylaxis of a hyaluronan-associated and/or proteoglycan-associated disease or disorder. The inventors have found out that the inventively obtainable purified modified bacterial hyaluronidase, preferably d016 and variants thereof, is in particular suitable for use in the treatment or prophylaxis of homozygous familial hypercholesterolemia (xanthomatose), heterozygous familial hypercholesterolemia, diabetic foot syndrome arterial hypertension or cardiac insufficiency.

Homozygous familial hypercholesterolemia (HoFH) is an inherited rare disease with a prevalence of 1 in 1,000,000. As a form of familial hypercholesterolemia, it is a lipid metabolism disorder. Patients with HoFH stand out due to a massive increase in low-density lipoprotein (LDL), a cholesterol fraction in the blood, with deposits in the skin and tendons, the so-called xanthomas (yellowish, nodular deposits of lipids). The lipid is also deposited in the vessel walls and causes early onset of severe atherosclerosis with a significantly reduced life expectancy. Patients suffer their first heart attack in early childhood, already from the age of about 5 years, have to undergo lipid apheresis weekly, receive concomitant medication in highest doses, and yet unfortunately have a life expectancy of only about 30 years on average.

HoFH is to be distinguished from the much more frequent heterozygous familial hypercholesterolemia (HeFH), which is also associated with a significantly increased risk of early cardiovascular events, but is less dramatic. The prevalence of this hereditary disease is estimated at 1:200 to 1:500. Here, too, signs of arteriosclerosis, i.e. diseases and symptoms caused by vascular disease, e.g. peripheral arterial occlusive disease, narrowing of the coronary arteries up to heart attack, stroke, can occur at a young age, but later and to a lesser extent than in the homozygous variant of the disease.

The inventively obtainable purified bacterial hyaluronidase is effective in the treatment and/or prophylaxis of HoFH and HeFH in view of its reducing effect on the tissue Extra Cellular Matrix (ECM) and chondroitin-6-sulfate and other proteoglycan levels in the atherosclerotic plaques and in the arteriosclerotic vessel walls. The administration of a therapeutically effective amount of the inventively obtainable purified modified bacterial hyaluronidase, preferably d016, thus leads to a size reduction of the stenosing plaques and an increase in the elasticity of the vascular wall.

Diabetic foot syndrome (DFS) is a syndrome of pathological changes based on painless sensory neuropathy and/or peripheral arterial occlusive disease (PAVK) in diabetes mellitus. It is most common in patients with type 2 diabetes mellitus and is associated with a high risk of poorly healing wounds on the foot. About 15% of diabetics develop painless (due to sensory neuropathy), poorly healing wounds on the feet in the course of their lives. Every year, about 4% of diabetics develop a new wound, and 0.1% develop a so-called Charcot foot due to the collapse of the arch of the foot.

In view of the PAVK aspect of the diabetic foot syndrome, the inventively obtainable purified bacterial hyaluronidase is effective in the treatment and/or prophylaxis of the diabetic foot syndrome by exhibiting a reducing effect on the tissue Extra Cellular Matrix (ECM) and chondroitin-6-sulfate and other proteoglycan levels in the atherosclerotic plaques and in the arteriosclerotic vessel walls. Thus, it leads to a size reduction of the stenosing plaques and an increase in the elasticity of the vascular wall. In addition, the inventive bacterial hyaluronidase has an effect on the neuropathic aspect of the diabetic foot syndrome. In this regard, it reduces the increased hyaluronic acid concentrations present in the nerve sheath regions caused by chronic polyneuritis. The associated nerve transmission disorder based on disturbed isolation function of the myelin sheaths is reduced and the nerve transmission ability is increased again.

In addition, the inventive pharmaceutical composition may be used in the treatment or prophylaxis of arterial hypertension and/or cardiac insufficiency. Without being bound to this theory, it appears that in response to an increased blood pressure due to arterial hypertension, the extracellular matrix of the blood vessels is enforced. This pressure-induced enforcement of the vascular extracellular matrix is inter alia effected by the increased presence of chondroitin-4-sulfate and/or chondroitin-6-sulfate chains. In the long run, the enforcement of the vasculature's extracellular matrix results in a hardening of the vessels which in turn leads to an increase in blood pressure. Hyaluronidase exerts its blood pressure-reducing effect by contributing to the breaking down of the excessively enforced vascular extracellular matrix and preferably, of the chondroitin-4-sulfate and/or chondroitin-6-sulfate chains which ultimately results in a more flexible vasculature and a decrease in blood pressure. In addition, Hyaluronidase preferably exerts its effect by hydrolyzing hyaluronic acid which is part of the backbone of the proteoglycans.

According to the present invention, arterial hypertension may be selected from the group consisting of endocrine hypertension, essential hypertension, arteriosclerotic hypertension, cardiovascular hypertension, renal hypertension, labile hypertension, neurogenic hypertension, paroxysmal hypertension, portal hypertension, pulmonary hypertension, and secondary hypertension. Also encompassed by “arterial hypertension” as used herein is a hypertension caused by monogenic defects such as glucocorticoid-remediable aldosteronism and Liddle's syndrome, hypertension caused by hypertension-susceptilibity genes such as angiotensinogen and alpha-adducin genes. “Arterial hypertension” according to the invention may as well be caused by environmental factors such as salt intake, preferably sodium intake, obesity, occupation, and alcohol intake, all of which forms of hypertension are also contemplated as being encompassed by the present invention. Moreover, hypertension in the sense of the present application also includes hypertension caused by obstructive sleep apnea, by aortic coarctation, by preeclampsia, by drugs such as combined oral contraceptive pill, cyclosporine, steroids, and by CNS disturbances.

Cardiac insufficiency may result from an ongoing or chronic arterial hypertension due to consecutive pressure load on the heart muscle. By lowering the blood pressure, hyaluronidase surprisingly allows for an effective treatment of cardiac insufficiency.

According to a preferred embodiment of the first inventive aspect, the nucleic acid sequence encoding bacterial SEQ ID No. 1 comprises or consists of SEQ ID No. 2. The nucleic acid sequence encoding the C-terminal HIS tag of the hyaluronidase of SEQ ID No. 1 is preferably SEQ ID No. 8 and the nucleic acid sequence encoding the N-terminal STREP tag of the hyaluronidase of SEQ ID No. 1 is preferably SEQ ID No. 6. The nucleic acid may be prepared according to any suitable method. An example method is described in the example section 1.1.1 below.

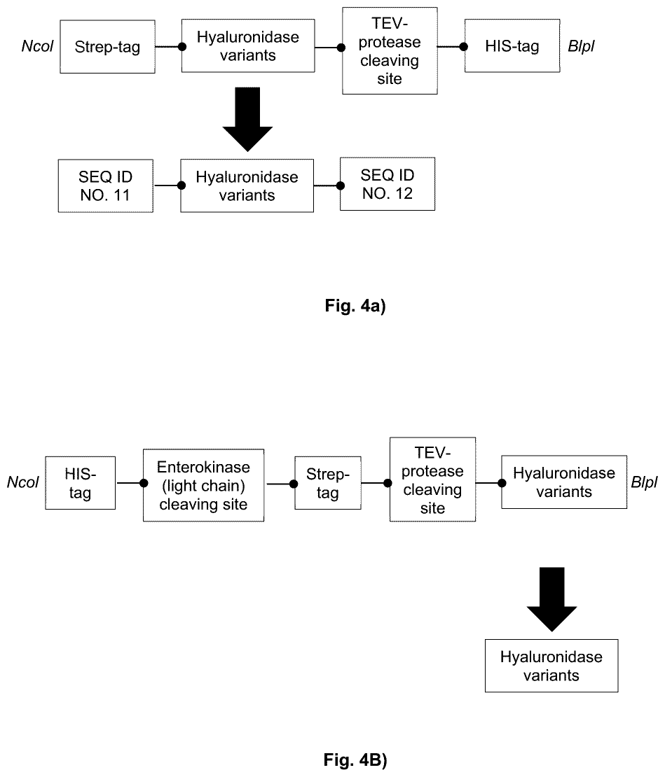

In addition, the inventive modified bacterial hyaluronidases may be obtained according to the first inventive aspect in that the DNA encoding the hyaluronidase in the expression vector comprises a cleavage site, in particular a Tobacco Etch Virus (TEV) protease cleavage site, e.g. of SEQ ID No. 15 between the hyaluronidase sequence and the C-terminal tag, preferably the HIS tag (see in particular FIG. 4a). The sequence may additionally comprise the N-terminal Ncol cloning sequence, e.g. of SEQ ID No. 13 (skipped sequence, consisting of CCATGGGC), and/or the C-terminal Blpl cloning sequence, e.g. SEQ ID No. 14 (skipped sequence in sequence listing consisting of GCTGAGC, for the respective expression vectors. After removing the C-terminal purification tag, such as the HIS tag, the resulting inventive modified bacterial hyaluronidase may comprise the N-terminal purification tag, e.g. as SEQ ID No. 11, and remaining C-terminal cleavage residues, e.g. of SEQ ID No. 12.

In the context of the present invention, the expression “nucleic acid” may refer to a nucleic acid encoding the modified bacterial hyaluronidase of SEQ ID No. 1, preferably d016tag. The nucleic acid preferably comprises or consists of at least 90% sequence identity to SEQ ID. 2, preferably the inventive nucleic acid consists of 100% SEQ ID No. 2.

Alternatively, “nucleic acid” refers to “a nucleic acid having at least 90% identity to any one of SEQ ID Nos: 19, 21, 23, 25, or 27 encoding a modified bacterial hyaluronidase polypeptide”, which means that means that the nucleic acid comprises at least 90, 91, 92, 93, 94, 95, 96, 97, 98, 99, 99.5, 99.6, 99.7, 99.8, 99.9%, or that it consists of 100% of the respective sequences of SEQ ID Nos: 19, 21, 23, 25, or 27.

Alternatively, “nucleic acid” refers to “a nucleic acid having at least 90% identity to any one of SEQ ID Nos. 29, 31, 33, 35, or 37 encoding a modified bacterial hyaluronidase polypeptide”, which means that means that the nucleic acid comprises at least 90, 91, 92, 93, 94, 95, 96, 97, 98, 99, 99.5, 99.6, 99.7, 99.8, 99.9%, or that it consists of 100% of the respective sequences of SEQ ID Nos. 29, 31, 33, 35, or 37.

The recombinant expression vector may be prepared according to any suitable method. An example method is described in example section 1.1.1 below. The inventive recombinant expression vector comprises the nucleic acid comprising or consisting of SEQ ID No. 2, any suitable vector, such as a pET-28a DNA vector, in particular pET28a using Ncol/Blpl restriction sites. The recombinant expression vector may be of any suitable form, such as in form of a plasmid. The DNA encoding the inventive modified bacterial hyaluronidase may contain a suitable N-terminal cloning site, such as a cloning site of SEQ ID No. 13, and a suitable C-terminal cloning site, such as a cloning site of SEQ ID No. 14. Using these, the DNA encoding sequences can be cloned into the suitable vector, such as pET28a(+) to enable expression in E. coli BL21(DE3).

The host cell transformed with the recombinant expression vector may be prepared according to any suitable method. An example method is described in example section 1.1.2 below. The inventive host cell may be selected from any suitable host cells. Preferably, the host cell is selected from E. coli cells, more preferably E. coli BL21(DE3) competent cells, which provide a comparatively high yield of modified bacterial hyaluronidase, preferably d016tag.

The inventive production process can be conducted in laboratory scale using conventional shaker flasks or may be up-scaled for use in a fermenter. In view of up-scaling to fermenter production, the process steps may be optimized in view of high density growth, onset/offset of expression, mixing speed and time, and applicable temperature. The inventors found out that in view of the high specific activity of the inventively obtainable purified modified bacterial hyaluronidase, preferably d016, already the laboratory scale using shaker flasks is sufficient to produce a meaningful amount (yearly amount) of the inventively obtainable purified modified bacterial hyaluronidase, preferably d016, for use in the treatment and/or prophylaxis of homozygous familial hypercholesterolemia (xanthomatose), heterozygous familial hypercholesterolemia, diabetic foot syndrome, arterial hypertension or cardiac insufficiency.

The inventive production process comprises or consists of the following steps:

Step a) Culturing a transformed host cell with recombinant expression vector comprising or consisting of a nucleic acid encoding a modified bacterial hyaluronidase polypeptide in a suitable growth medium under suitable growth conditions to express the bacterial hyaluronidase polypeptide comprising or consisting of at least 90% sequence identity to SEQ ID No. 1, wherein the hyaluronidase polypeptide comprises a C-terminal tag and an N-terminal tag, The N-terminal tag may be a STREP tag and/or the C-terminal tag may be a HIS tag. As an example, terrific broth (TB) media can be used. The respective broth media may be supplemented by suitable antibiotics, such as kanamycin, and/or buffer constituents, such as potassium phosphate buffer. The growth media also comprises a suitable expression inducer, such as Isopropyl β-D-1-thiogalactopyranoside (IPTG). According to laboratory scale, cell growth and protein expression may be conducted in a shaker culture flask at a suitable temperature, preferably at 28 to 30° C. and shaking, preferably 180 rpm, for 18 to 20 hours.

Alternatively, step a) may be performed in that a transformed host cell is cultured with a recombinant expression vector comprising or consisting of a nucleic acid of at least 90% sequence identity to any one of SEQ ID Nos: 19, 21, 23, 25, or 27 encoding a modified bacterial hyaluronidase polypeptide in a suitable growth medium under suitable growth conditions to express the bacterial hyaluronidase polypeptide, wherein the hyaluronidase polypeptide comprises a C-terminal tag and an N-terminal tag.

Alternatively and in particular with respect to the fifth inventive aspect, step a) may be performed in that a transformed host cell is cultured with a recombinant expression vector comprising or consisting of a nucleic acid of at least 90% sequence identity of any one of SEQ ID Nos. 29, 31, 33, 35, or 37 encoding a modified bacterial hyaluronidase polypeptide in a suitable growth medium under suitable growth conditions to express the bacterial hyaluronidase polypeptide, wherein the bacterial hyaluronidase polypeptide comprises a N-terminal tag including at least a first N-terminal purification tag connected by a cleavage site to a second different purification tag which is connected to the hyaluronidase polypeptide either directly or by a second cleavage site,

Step b) Harvesting the cultured transformed host cell of step a). After culturing the transformed host cells under suitable growth conditions, the host cells are harvested with suitable methods. According to laboratory scale, preferably pyrogen-free, sterile tubes are used to collect the harvested host cell containing growth media. This harvested growth medium is preferably centrifuged, preferably at 4 000 rcf at a reduced temperature, preferably 4° C., for at least 30 minutes in order to separate the medium from the host cells, which aggregate to so called pellets after centrifugation. The supernatant is to be discarded and the harvesting tubes are preferably sealed and stored at reduced temperature, preferably below 0° C., more preferably at −80° C.

Step c) Lysing the harvested host cells of step b) and separating resulting host cell fragments from resulting host cell content comprising the bacterial hyaluronidase polypeptide. According to laboratory scale, the host cells, which after centrifugation form aggregated pellets, are generally resuspended and mixed, preferably by vortexing, in suitable medium, preferably a suitable buffer medium, such as phosphate buffered saline (PBS; containing 280 mM NaCl, 6 mM KCl, 15.1 mM Na₂HPO₄, 4.9 mM NaH₂PO₄, PH=7.4 at room temperature). The host cells are lysed by any suitable methods, such as sonication. In order to maintain suitable temperatures within the medium, the sonication takes preferably place at reduced temperatures, more preferably wherein the tubes are surrounded by ice while sonicating the medium. The resulting host cell fragments are separated from the inventive bacterial hyaluronidase polypeptide with suitable methods, preferably by centrifugation, more preferably centrifugation at reduced temperature, e.g. 4° C., for at least 30 minutes at e.g. 4 000 rcf. The supernatant comprising the bacterial hyaluronidase is preferably transferred to a new tube and optionally one or more centrifugation steps are further conducted. The resulting supernatant comprising the bacterial hyaluronidase including the C- and N-terminal tags is then used for the purification step d).

Step d) Purifying the resulting host cell content of step c) with HIS affinity chromatography and STREP affinity chromatography to result in a purified bacterial hyaluronidase polypeptide including the C- and N-terminal HIS- and STREP tags. Preferably the HIS- and STREP affinity chromatography takes place subsequently, wherein the order is interchangeable, i.e, wherein the HIS affinity chromatography purification is conducted first followed by STREP affinity chromatography or vice versa. In the following the subsequent purification conducting first HIS affinity chromatography and then STREP affinity chromatography is described as one example embodiment. In view that HIS affinity chromatography columns are generally cheaper than STREP affinity chromatography columns, the high purity yield may be achieved in a more cost effective way in case the HIS affinity chromatography is conducted first.

According to the present invention, any suitable HIS affinity chromatography can be used in order to bind to the C-terminal HIS tag (amino acids 735 to 740 of SEQ ID NO. 1, see also SEQ ID NO. 7) of the inventive modified bacterial hyaluronidase. According to laboratory scale, as an example one or more suitable gravitational HIS-purification columns are equilibrated to general PBS downstream buffer. The content of each tube resulting after step c) is distributed to the one or more HIS-columns. Optionally the HIS purification procedure is repeated one, two or more times, preferably two times. The loaded one or more HIS-columns are then preferably washed with a suitable washing medium, e.g. with 10 mM imidazole PBS solution, before being eluted in a suitable eluting medium, e.g. 150 mM imidazole PBS solution. Generally, the eluate of the one or more HIS-columns originating from the same production flask (tube) are combined and preferably diluted to 35 ml with general downstream buffer PBS. Preferably, the purified eluate samples comprising the modified bacterial hyaluronidase including HIS- and STREP tag is then stored under reduced temperature, preferably on ice until the following STREP purification.

According to the present invention, any suitable STREP affinity chromatography can be used in order to bind to the N-terminal STREP tag (amino acids 3 to 10 of SEQ ID NO. 1, see also SEQ ID NO. 5) of the modified bacterial hyaluronidase including HIS- and STREP tag. According to laboratory scale, one or more suitable syringe-based STREP affinity chromatographic columns can preferably be used. As an example, one or more syringe-based STREP-purification steps, more preferably with a flowrate <5 ml/min, are performed. STREP-columns (5 ml bed volume) are generally washed and equilibrated with 2×25 ml general downstream buffer PBS. A suitable amount of the eluate sample resulting from the HIS purification is applied and runs through the STREP column. The loaded STREP column is then preferably washed with a suitable amount of general downstream PBS buffer to remove any remaining contaminant proteins. By applying a suitable eluting medium, e.g. 2.5 mM d-Desthiobiotin containing PBS buffer, the protein of interest, namely the modified bacterial hyaluronidase including HIS- and STREP tag, is eluted, preferably into a fresh, sterile and pyrogen-free tube. This tube is either stored at reduced temperature, e.g. on ice, or the lyophilized to result in a dry storable product of the purified modified bacterial hyaluronidase including HIS- and STREP tag. The STREP column can be reused after suitable regeneration according to the prior art.

Optionally, the buffer of the eluate may be exchanged by suitable methods including centrifugation of the eluate to aggregate the inventive bacterial hyaluronidase, discarding the supernatant and resuspending the aggregated inventive bacterial hyaluronidase into a different buffer medium, such as Tris-HCL NaCl. According to a preferred embodiment the resuspended bacterial hyaluronidase including HIS- and STREP tag is stored under reduced temperature, e.g. on ice for further post processing, such as polishing and removal of at least part of the HIS-tag and/or the STREP tag using the respective cleavage sites as discussed above.

According to a preferred embodiment, the inventive purification step additionally comprises one or more suitable polishing steps to remove last impurities of the inventive bacterial hyaluronidase polypeptide in step d) and, thus, to increase purity thereof. According to laboratory scale, one or more endotoxin removal steps, such as Polymyxin B based endotoxin removal steps; one or more sterile filtration steps, and one or more particle removal steps can be performed.

According to the first aspect of the present invention, at least part of the C-terminal and/or N-terminal tag is removed from the purified bacterial hyaluronidase of step d) to result in the inventively obtainable purified modified bacterial hyaluronidase, preferably of a sequence of at least 90% sequence identity to SEQ ID Nos. 9, 10, 20, 22, 24, 26, 28, 30, 32, 34, 36, or 38, wherein the sequence includes no, one, two or three amino acid residues at the C-terminus, preferably wherein the amino acid residues are respectively histidine residues, and no, one, two, three, or more residues at the N-terminus, preferably wherein the one, two, three or more amino acid residues are preferably selected from the STREP tag of SEQ ID No. 5, and wherein such a inventively obtainable purified bacterial hyaluronidase is also synonymously called “d016” with respect to SEQ ID Nos. 9, 10, 20 or 30, and wherein the inventively obtainable purified bacterial hyaluronidase is also synonymously called “d016 variant” with respect to SEQ ID Nos. 22, 24, 26, 28, 32, 34, 36, or 38.

According to an embodiment of the present invention, the purification tags may be removed from the hyaluronidase polypeptide using a suitable cleaving protease, such as a Tobacco Etch Virus (TEV)-protease as discussed above (SEQ ID Nos. 15 and 16), or an enteropeptidase as discussed above (SEQ ID Nos. 17 and 18). In case the HIS-tag is connected to the hyaluronidase sequence using a TEV-protease cleaving site sequence, the HIS-tag may accordingly be removed using a respective TEV protease. Accordingly, the resulting inventive modified bacterial hyaluronidase of at least 90% sequence identity of SEQ ID Nos. 20, 22, 24, 26, or 28 may comprise a sequence of SEQ ID No. 11 as N-terminal residues and a sequence of SEQ ID No. 12 as C-terminal residues. The inventive modified bacterial hyaluronidase of at least 90% sequence identity of SEQ ID Nos. 30, 32, 34, 36, or 38 may preferably comprise only the hyaluronidase sequence and no remaining residues of the previously included N-terminal tags.

According to a further additional or alternative embodiment, the inventive hyaluronidase peptide may be a modified hyaluronidase peptide with increased lipophilic constituents, and/or increased positively charged constituents, and/or an amino acid sequence binding to at least part of a blood-brain barrier transporter system, such as to large neutral amino acid receptor, in particular L-type Amino acid transporter (LAT1), and/or with an amino acid sequence binding to at least part of a transferrin receptor, such as transferrin receptor 1 or transferrin receptor 2.

According to the present invention, the laboratory scale protein yield results in 0.09 to 0.13 mg/ml after HIS purification and 0.04 to 0.06 mg/ml after dual HIS/STREP purification and subsequent polishing purification step (see example section item 1.3.4 below). It is expected that this yield significantly increases upon fermentation scale production.

According to the second aspect of the present invention, an inventively modified bacterial hyaluronidase polypeptide obtainable according to the inventive production process according to the first and fifth aspect is provided. In an alternative, the second aspect of the present invention provides a modified bacterial hyaluronidase polypeptide consisting of 99.5%, 99.6, 99.7, 99.8, 99.9, 100% sequence identity to any one of SEQ ID Nos. 9, 10, 20, 22, 24, 26, 28, 30, 32, 34, 36, or 38. All features and embodiments disclosed with respect to the first and fifth aspect of the present invention are combinable alone or in (sub-) combination with the second aspect of the present invention including each of the preferred embodiments thereof, provided the resulting combination of features is reasonable to a person skilled in the art.

According to a third aspect of the present invention, a pharmaceutical composition comprising the inventively obtainable modified bacterial hyaluronidase polypeptide according to the first and fifth aspect of the present invention or the inventive modified bacterial hyaluronidase polypeptide of the second aspect of the present invention in a therapeutically effective amount and one or more pharmaceutically acceptable excipients is provided. All features and embodiments disclosed with respect to the first, second and fifth aspect of the present invention are combinable alone or in (sub-) combination with the third aspect of the present invention including each of the preferred embodiments thereof, provided the resulting combination of features is reasonable to a person skilled in the art.

In general, the therapeutically effective amount of the inventively obtainable bacterial hyaluronidase depends on the therapeutic application of the pharmaceutical composition. According to the present invention, the term “therapeutically active amount” means that the amount of inventively obtainable or the inventive modified bacterial hyaluronidase polypeptide, preferably wherein the amount of the modified bacterial hyaluronidase polypeptide comprising or consisting of at least 90% sequence identity of any one of SEQ ID Nos. 9, 10 20, 22, 24, 26, 28, 30, 32, 34, 36, or 38 in the pharmaceutical composition or in a pharmaceutical unit dose thereof is suitable for treatment or prophylaxis of a hyaluronan-associated and/or proteoglycan-associated disease or disorder, preferably selected from a group consisting of homozygous familial hypercholesterolemia (xanthomatose), heterozygous familial hypercholesterolemia, diabetic foot syndrome, arterial hypertension or cardiac insufficiency.

In case the inventive pharmaceutical formulation needs to pass the blood-brain barrier, the inventive pharmaceutical composition is preferably simultaneously or sequentially administered with one or more blood-brain barrier efflux inhibitory agents, preferably selected from the group consisting of p-glycoprotein inhibitory agents, such as Cyclosporine or a Cyclosporine derivative, such as Valspodar, Elacridar, Zosuquidar; calcium antagonistic agents, such as Verapamil; procyanidine or procyanidine derivatives; etc. In case of sequential administration, the blood-brain barrier efflux inhibitory agent is administered prior to the hyaluronidase peptide. Furthermore, the inventively obtainable/inventive modified bacterial hyaluronidase sequences of the first and fifth/second aspect is preferably selected from SEQ ID Nos. 22, 24, 26, 28, 32, 34, 36, or 38 in order to increase the transmission over the blood-brain barrier, preferably without the additional administration of a blood-brain barrier efflux inhibitory agent.

As an example, in case the pharmaceutical composition is a parenteral liquid composition for intravenous application, the inventive bacterial hyaluronidase may be provided as a concentrate in a vial with 1 to 10, preferably 2 or 5 mL in a therapeutically effective amount between 15,000 to 1,500,000 U/mL. According to a preferred embodiment, the unit dose of the inventive pharmaceutical composition comprises the inventive modified bacterial hyaluronidase polypeptide in a concentration range of 200 U per kg/per day to 30,000 U per kg/per day. In order to increase the half-life of the inventive bacterial hyaluronidase, the dosing scheme may preferably comprise a suitable bolus amount of the inventive hyaluronidase in order to saturate the exo- and/or endo-proteinases followed by a subsequent administration of the therapeutically effective unit dose amount. Preferably, in case of intravenous administration, the subsequent unit dose is administered up to 1 hour, alternatively up to 30 minutes or up to 15 minutes or up to 5 minutes after the bolus administration of the inventive bacterial hyaluronidase. Alternatively, in case the inventive modified bacterial hyaluronidase sequences shall cross the blood-brain barrier, the hyaluronidase may be administered in two consecutive applications, wherein the second unit dose is administered up to 12 hours, preferably 1 to 6 hours after the first administration of the inventive modified bacterial hyaluronidase. More preferably, the unit dose may comprise 10,000 U, 11,000 U, 12,000 U, 13,000 U, 14,000 U, 15,000 U or more inventive modified bacterial hyaluronidase. It is presently believed, that the first administration of the inventively obtainable/inventive hyaluronidase, in particular of a high dose of inventively obtainable/inventive hyaluronidase may condition the blood-brain barrier in such a way that it the second subsequent administration of hyaluronidase shows an increased uptake.

Notwithstanding the aforementioned, the inventive pharmaceutical composition can be present in any suitable application form, such as solid, semi-solid or liquid application form. The therapeutically effective amount is to be calculated accordingly. As an example, the solid form of inventive pharmaceutical composition may be presented as dried or lyophilized form. In addition to the inventive bacterial hyaluronidase polypeptide, one or more pharmaceutically acceptable excipients, such as one or more constituents selected from the group of bulking agents, buffering agents, tonicity modifiers, collapse temperature modifiers, solvents and/or co-solvents, solubilizing agents, preservatives, antioxidants, antimicrobial and chelating agents, wetting agents, flocculating/suspending agents, and optionally one or more proteinase inhibitors, such as selected from metallo-proteinase inhibitors, dipeptidyl-4 exopeptidase inhibitors (syn: DPP-4 inhibitors or gliptins), or hyaluronan binding protein 2 protease inhibitors, can be comprised.

According to the present invention, suitable bulking agents may comprise sucrose, lactose, trehalose, mannitol, sorbitol, glucose, raffinose, glycine, histidine or polyvinylpyrrolidone (K40). Suitable buffering agents may comprise sodium citrate, sodium phosphate, sodium hydroxide, Tris base 65, Tris acetate, or Tris HCl 65. A suitable tonicity modifier may comprise dextrose. Suitable collapse temperature modifier may be dextran, ficoll, gelatin, hydroxyethyl starch. Suitable solvents are preferably selected from water for injection, and non-aqueous water miscible agents, such as ethanol, glycerin, propylene glycol and n-lactamide, may be used as co-solvents. Suitable solubilizing agents may be selected from suitable surfactants and co-solvents. Few examples of suitable surfactants are Polyoxyethylene sorbitan monooleate (Tween 80), Sorbitan monooleate, Polyoxyethylene sorbitan monolaurate (Tween 20), Lecithin, Polyoxyethylene polyoxypropylene copolymers (Pluronics). Examples of suitable co-solvents as solubilizing agents are Propylene glycol, Glycerin, Ethanol, Polyethylene glycol (300 and 400), Sorbitol, Dimethylacetamide and Cremophor EL. Suitable preservatives may be selected from parabens, such as Benzyl alcohol (0.9% to 1.5%), Methylparaben (0.18% to 0.2%), Propylparaben (0.02%), Benzalkonium chloride (0.01% to 0.02%), and Thiomersal (0.001% to 0.01%). Suitable antioxidants are preferably selected from Ascorbic acid, Sulfurous acid salts, such as Sodium bisulite, Sodium meta and bisulfite, Sodium formaldehyde sulfoxylate, Thiourea, Acetylcystein, Ascorbic acid ester, butylated hydroxy toluene, tocopherols. Suitable antimicrobial agents are selected from Phenol, Meta-cresol, Benzyl alcohol, Parabens (methyl, propyl, butyl), Benzalkonium chloride, Chlorobutanol, Thimerosal, Phenylmercuric salts (acetate, borate, nitrate). Suitable chelating agents are selected from ethylene diamine tetra acetic acid salt. Suitable wetting agents are preferably selected from glycerin, alcohol and propylene glycol. Suitable flocculating/suspending agents are selected from electrolytes, such as potassium/sodium chloride, potassium/sodium citrate or potassium/sodium acetate, or surfactants and hydrophilic colloids, such as sodium carboxymethyl cellulose, acacia, gelatin, methyl cellulose, polyvinyl pyrrolidone. The proteinase inhibitors may increase the half-life of the inventive bacterial hyaluronidase and, thus, may reduce the total amount of hyaluronidase to be used or may increase the therapeutic efficacy. Edetate Calcium Disodium may be used to broadly inhibit metalloproteases. Vildagliptin or Linagliptin may be used to inhibit specifically DPP-4 exopeptidase, capable of cutting sequences with Proline. Aproptinin may be suitable to inhibit hyaluronan binding protein 2 (HABP2) and may be suitable to inhibit a broad range of serine proteases.

In case of lyophilized pharmaceutical compositions, the one or more excipients may be selected from the suitable bulking agents, buffering agents, tonicity modifiers, collapse temperature modifiers, and proteinase inhibitors. In case the inventive pharmaceutical composition is applied as a parenteral injection, the one or more pharmaceutical excipients may be selected from solvents, solubilizing agents, co-solvents, preservatives, wetting agents/surfactants, flocculating/suspending agents and proteinase inhibitors.

The respective one or more proteinase inhibitors may alternatively be administered separately to the inventive pharmaceutical composition comprising the inventive bacterial hyaluronidase. In case of separate administration, the one or more suitable proteinase inhibitors are generally administered in a suitable amount to effectively inhibit exo- and/or end-peptidases prior to or concomitantly with the inventive bacterial hyaluronidase.

In case, the inventive pharmaceutical composition including the inventive hyaluronidase is administered in form of a bolus administration/first administration followed by a subsequent unit dose administration, the protease inhibitor agents may be comprised in the bolus and optionally the subsequent unit dose of the inventive pharmaceutical composition. In order to reduce the body burden of proteinase inhibitors, preferably only the bolus administration comprises the suitable one or more protease inhibitors or the separate administration of the one or more protease inhibitors is administered prior to or concomitantly to the bolus administration of the inventive pharmaceutical composition including the inventive hyaluronidase.

As an alternative example, the liquid form of inventive pharmaceutical composition may be presented as a suspension of the inventive bacterial hyaluronidase polypeptide in a suitable suspension medium, preferably in a suitable suspension medium for parenteral application, more preferably for intravenous application. The pharmaceutical composition may be in form of a concentrate, which is to be diluted prior to parenteral application. The pharmaceutical composition for parenteral injection may comprise one or more excipients selected from the group of solubilizing agents, co-solvents, preservatives, wetting agents/surfactants, flocculating/suspending agents and proteinase inhibitors.

As an example, the liquid form of the inventive pharmaceutical composition (after dilution) for parenteral injection comprises 0.9% NaCl solution, Ringer solution, or sodium lactate-sodium chloride solution.

In general, the inventive pharmaceutical composition can be suitable for per oral, nasal, transdermal, rectal, intravenous, or intramuscular application. In case of per oral application, the inventive bacterial hyaluronidase polypeptide is preferably formulated with a suitable enteric coating to avoid/reduce degradation of the inventive bacterial hyaluronidase in enteric fluids. Particularly preferred is the intravenous application of the inventive pharmaceutical composition, as the inventive bacterial hyaluronidase polypeptide is directly, without first pass effects, present in the vascular space, which is preferably in particular for use in the treatment or prophylaxis of a hyaluronan-associated and/or proteoglycan-associated disease or disorder, preferably selected from a group consisting of homozygous familial hypercholesterolemia (xanthomatose), heterozygous familial hypercholesterolemia, diabetic foot syndrome, arterial hypertension or cardiac insufficiency. In this case, the exo- and endo-peptidases may be inhibited by use of a bolus application of the inventive bacterial hyaluronidase and/or the additional administration of proteinase inhibitors in the same pharmaceutical composition or in a separate pharmaceutical composition. Alternatively, the nasal application is furthermore preferred, as the first pass effect is also circumvented.

A method of treating a hyaluronan-associated and/or proteoglycan-associated disease or disorder, preferably selected from a group consisting of a homozygous familial hypercholesterolemia (xanthomatose), heterozygous familial hypercholesterolemia, diabetic foot syndrome, arterial hypertension or cardiac insufficiency comprising or consisting of administering the inventively obtainable modified bacterial hyaluronidase polypeptide according to the second aspect, or the inventive pharmaceutical composition according to the third aspect to a subject in need thereof.

According to the fifth inventive aspect (also an alternative to the first inventive aspect), a process of production of a purified modified bacterial hyaluronidase polypeptide of at least 90% sequence identity of any one of SEQ ID Nos. 9, 10, 30, 32, 34, 36, or 38 comprising or consisting of the following steps is provided:
a. Culturing a transformed host cell with recombinant expression vector comprising or consisting of a nucleic acid of at least 90% sequence identity of any one of SEQ ID Nos. 29, 31, 33, 35, or 37 encoding a modified bacterial hyaluronidase polypeptide in a suitable growth medium under suitable growth conditions to express the bacterial hyaluronidase polypeptide, wherein the bacterial hyaluronidase polypeptide comprises a N-terminal tag including at least a first N-terminal purification tag connected by a cleavage site to a second different purification tag which is connected to the hyaluronidase polypeptide either directly or by a second cleavage site,
b. Harvesting the cultured transformed host cell of step a),
c. Lysing the harvested host cells of step b) and separating resulting host cell fragments from resulting host cell content comprising the bacterial hyaluronidase polypeptide, and
d. Purifying the resulting host cell content of step c) with a first affinity chromatography corresponding to the N-terminal first purification tag, removing the first purification tag from the remaining hyaluronidase sequence and purifying the resulting hyaluronidase sequence with a second affinity chromatography corresponding to the second purification tag and optionally removing the N-terminal second purification tag to result in a purified form of the bacterial hyaluronidase polypeptide comprising or consisting of at least 90% sequence identity to SEQ ID Nos. 9, 10, 30, 32, 34, 36, or 38.

The production process embodiments of the first inventive aspect can also be used with respect to the production process of the fifth inventive process. Accordingly, the host cell can generally be E. coli, preferably E. coli BL21(DE3). The DNA encoding the inventive modified bacterial hyaluronidase (See also FIG. 4b) may contain a suitable N-terminal cloning site, such as a cloning site of SEQ ID No. 13, and a suitable C-terminal cloning site, such as a cloning site of SEQ ID No. 14. Using these, the DNA encoding sequences can be cloned into the suitable vector, such as pET28a(+) to enable expression in E. coli BL21(DE3).

Furthermore, the purification tags may be selected from any suitable sequences, preferably from a HIS tag and Strep tag, in particular wherein the first purification N-terminal tag is a HIS tag, preferably a HIS tag of SEQ ID No. 7, and/or wherein the N-terminal second purification tag is a STREP tag, preferably a STREP tag of SEQ ID No. 5. According to a further embodiment of the present invention, the cleaving sites represent DNA encoding protease cleaving sites, such as a Tobacco Etch Virus (TEV) protease cleaving site (SEQ ID No. 15) and/or an enteropeptidase cleaving site, such as an enterokinase (light chain) cleaving site (SEQ ID No. 17). The resulting inventive modified bacterial hyaluronidase is preferably free of remaining residues of the N-terminal sequence of purification tags and cleaving sites.

The further production process embodiments of the first aspect and also set out in the following example section can be used for carrying out the production process of the fifth inventive aspect, provided the resulting combination of features is reasonable to a person skilled in the art.

The present invention is described in the following on the basis of exemplary embodiments, which merely serve as examples and which shall not limit the scope of the present protective right.

### EXAMPLES

Further characteristics and advantages of the present invention will ensue from the following description of example embodiments of the inventive aspects with reference to the accompanying drawings.

All of the features disclosed hereinafter with respect to the example embodiments and/or the accompanying figures can alone or in any sub-combination be combined with features of the aspects of the present invention including features of preferred embodiments thereof, provided the resulting feature combination is reasonable to a person skilled in the art.

### Equipment (General)

1. Thermo Scientific Multifuge X3R (SN: 42343259)
2. Eppendorf Centrifuge 5920R (SN: 5948HR902433)
3. New Brunswick Scientific Innova 4300 (SN: 590544115)
4. New Brunswick Scientific Innova 4300 (SN: 791060864)
5. Jenway 6305 Spectrophotometer (SN: 68993)
6. ARCTIKO ULTF 420-80° C. Freezer (SN: 20180262153)
7. Liebherr GX 823-20M Freezer (SN: 50.636.690.7)
8. Sartorius Arium Mini UV Ultrapure Water (SN: 36802288)
9. VWR Vapour Line 135-B Autoclave (MN: 12175020)
10. Peqlab TS-100 Thermoshaker (SN: 430805037) with 24×2 ml block (SN: 191165)
11. IKA Vortex 2 S000 (SN: 100451198)
12. Fisher Scientific Mini300V Plus Power Supply (SN: 190118120)
13. IKA Rocker 2D basic Shaker (SN: 100574254)
14. Kern Precision Balance ABJ320-4NM (SN: WB18AM0063)
15. Kern PBS4200-2M Balance (SN: WB17M0023)
16. AQUALYTIC pH-Meter SD AL 10 PH (SN: AJ. 12842)
17. Eppendorf 500-5000 μl pipette (SN: G441391)
18. Eppendorf 100-1000 μl pipette (SN: J37828B)
19. Eppendorf 100-1000 μl pipette (SN: H41815D)
20. Eppendorf 10-100 μl pipette (SN: H39057D)
21. Eppendorf 10-100 μl pipette (SN: H39145D)
22. Eppendorf 0.1-10 μl pipette (SN: 400555A)
23. Bandelin Sonoplus GM 2200.2 Generator (SN: 3714.00125432.005) with Converter UW 2200 (SN: 599.00125433.006) and KE76 horn
24. Epson Workforce Pro WF-4720 (model C582A) (SN: *X2TU046376*)
25. Thermo Scientific NanoDrop Lite (SN: 5149)

### Materials/Chemicals (General)

1. Glycerin (CAS: 56-81-5) s
2. Agar-Agar (CAS: 9002-18-0)
3. Yeast Extract (CAS: 8013-01-2)
4. Isopropyl β-D-1-thiogalactopyranoside IPTG (CAS: 367-93-1)
5. Vegetal Peptone (CAS: 91079-46-8)
6. Sodium Chloride NaCl (CAS: 7647-14-5)
7. Potassium Chloride KCl (CAS: 7447-40-7)
8. Kanamycin solution (CAS: 25389-94-0)
9. Sodium Phosphate monobasic NaH2PO4 (CAS: 13472-35-0)
10. Sodium Phosphate dibasic Na2HPO4 (CAS: 7558-79-4)
11. Potassium Phosphate monobasic KH2PO4 (CAS: 7778-77-0)
12. Potassium Phosphate dibasic K2HPO4 (CAS: 7758-11-4)
13. Imidazole (CAS: 288-32-4)
14. d-Desthiobiotin (CAS: 533-48-2)
15. Hydrochloric Acid HCl (CAS: 7647-01-0)
16. Sodium Hydroxide NaOH (CAS: 1310-73-2)
17. Tris base (CAS: 77-86-1)
18. His GraviTrap TALON (Sigma-Aldrich GE29-0005-94)
19. StrepTrap™ High Performance (Sigma-Aldrich GE28-9075-47)
20. Bovine Serum Albumin BSA (CAS: 9048-46-8)
21. Hyaluronic Acid (CAS: 9067-32-7)
22. Sodium Acetate NaOAc (CAS: 6131-90-4)
23. 2× LAEMMLI buffer (beta-mercaptoethanol CAS: 60-24-2, Sodium Dodecyl Sulfate CAS: 151-21-3)
24. Tris-MOPS SDS Running buffer (Sodium Dodecyl Sulfate CAS: 151-21-3, Tris CAS: 77-86-1, EDTA CAS: 60-00-4, MOPS CAS: 1132-61-2)
25. Methanol MeOH (CAS: 67-56-1)
26. Comassie brilliant blue R-250 (CAS: 6104-59-2) 27. Bovine Hyaluronidase Typ I-S(CAS: 37326-33-3)

### 1: Production of the Inventive Modified Bacterial Hyaluronidase Polypeptide of SEQ ID 1

### 1.1 Preparation

1.1.1 Gene Synthesis and Subcloning—pET28a(+)

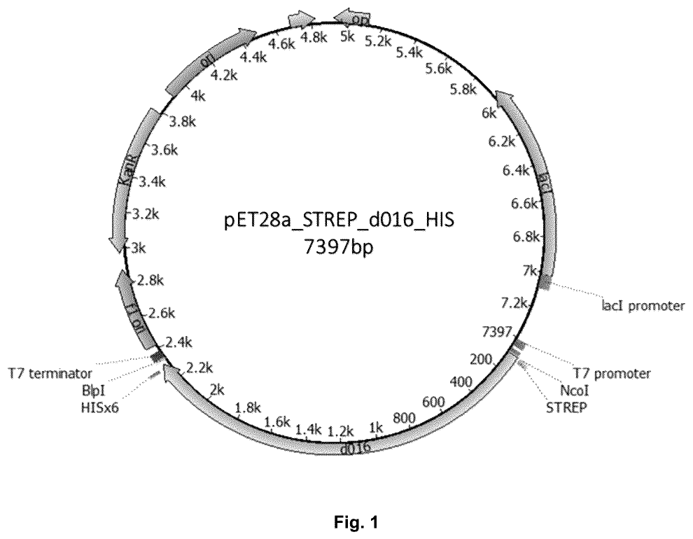

The d016tag gene sequence of SEQ ID No. 2 encoding the inventive modified bacterial hyaluronidase of SEQ ID No. 1 was designed by truncation of the wildtype sequence of Streptococcus pneumonia of SEQ ID No. 4 and the addition of C-terminal HIS-Tag gene sequence of SEQ ID No. 8 and N-terminal Strep-Tag gene sequence of SEQ ID No. 6 for a two-step affinity chromatography purification. By design, both tags were designated to remain on the final enzyme product for increased solubility and protection against (exo-) peptidases in the vascular space, which is the designated drug compartment. Synthesis of the gene construct, cloning into pET28a using Ncol/Blpl restriction sites leads to a recombinant expression vector (syn.: plasmid comprising pET28a(+) vector including d016tag insert). The Vector Map Plasmid of pET28a(+) and insert d016tag is displayed in FIG. 1.

1.1.2 Plasmid Transformation into—E. coli BL21(DE3)

The plasmid was transformed into host cells of E. coli BL21(DE3) competent cells via heat-shock at 42° C. according to supplier protocol (New England Biolabs). Selection for positive transformants of inventive host cells was performed on lysogeny broth (LB) agar plates with 50 μg/ml kanamycin antibiotics in accordance with pET28a encoded resistance. The plates were grown for 48 h at room temperature.

### 1.1.3 Transformant Cultivation of Inventive Host Cells in Lysogeny Broth (LB-Media and Glycerol Stock Preparation (−80° C. Storage)

A single clone of inventive host cells was picked from the plate and cultivated overnight at 37° C. in LB media with 50 μg/ml kanamycin. A sterile, pyrogen-free tube was prepared with sterilized 50% glycerol solution. Under sterile laminar flow conditions, the overnight grown liquid culture (12-14 h) was diluted in the master clone storage tube containing 50% liquid culture volume and 50% of prepared glycerol solution. The master clone was stored at −80° C. All batches of d016tag inventive host cell cultivation originate from this master clone.

### 1.2 Production

### 1.2.1 Pre-Culture Inoculation in LB-Media and Overnight Growth (37° C.)

Two 250 ml culture flask (autoclaved) were prepared with (vegetal) LB media (autoclaved). 50 μg/ml kanamycin was added to prevent growth of potential bacterial contaminants. The preparation/handling of the pre-culture was conducted under sterile laminar flow conditions at all times. One flask with 65 ml of kanamycin-containing (vegetal) LB-media was inoculated by a single pipette tip (autoclaved) of sample from the master clone storage tube. A second flask was used as negative control with a pipette tip not containing cells of the master clone. The preculture tubes were sealed by an air-permeable membrane under laminar flow and delivered to the incubation shaker. Cells were grown at 37° C. overnight (12-14 h) at 180 rpm. The master clone storage tube was tightly sealed throughout the process and only opened under laminar flow conditions. After inoculation, the master clone tube was sealed and stored at −80° C.

### 1.2.2 Main Culture Inoculation in Terrific Broth (TB) Media, Isopropyl β-D-1-Thiogalactopyranoside (IPTG) Induced Expression Overnight (28-30° C.)

Fourteen 500 ml baffled flasks with air-permeable screwcaps were autoclaved and each was filled with 200 ml of (vegetal) TB-media (autoclaved, vegetal peptone 12 g/l, yeast extract 24 g/l, glycerol 8 ml/l) containing potassium phosphate buffer (autoclaved separately, final concentration of 9.4 g/l K₂HPO₄ and 2.2 g/l KH₂PO₄). 50 μg/ml kanamycin was added to prevent growth of potential bacterial contaminants. 12 flasks were individually inoculated by 4 ml of the preculture using 5 ml pipette tips (autoclaved). The two remaining flasks containing 4 ml of the control preculture were used as controls. The preparation of the main cultures was performed under sterile laminar flow conditions at all times. All flask caps were tightly screwed before flasks were transferred to the incubation shaker, where growth conditions were set to 37° C., 180 rpm. After 3-4 h and a growth OD600 of 0.7-1.1, all flasks were equilibrated to room temperature. Protein production was induced with 200 μl of a 100 mM IPTG stock solution. Finally, all flasks (including contamination controls) were placed inside a second incubator shaker at 28-30° C. (180 rpm) for 18-20 h.

### 1.2.3 Culture Harvesting and Cell Pellet Storage (−80° C.)

After the defined growth period of 18-20 h, culture flasks were taken out of the incubation shakers. Control flasks were checked to ensure that no growth of contaminants had occurred within the batch. Controls were not processed further. All twelve culture flasks were harvested using pyrogen-free, sterile 50 ml tubes. 3×50 ml of each flask were harvested and the remaining volume was discarded. All tubes were centrifuged at 4000 rcf, 4° C. for 30-45 min. The supernatant was discarded, and all tubes containing pellet comprising the inventive modified bacterial hyaluronidase were tightly sealed and stored at −80° C. for use in downstream processing.

### 1.3 Downstream Processing

### 1.3.1 Harvesting/Preprocessing: Cell Lysis by Sonication, Fragment Removal by Centrifugation

Pellets were resuspended in 10 ml of the general downstream buffer PBS (phosphate buffered saline, containing 280 mM NaCl, 6 mM KCl, 15.1 mM Na₂HPO₄, 4.9 mM NaH₂PO₄, pH=7.4 at room temperature) by thorough vortexing. The three tubes of the same flask origin were combined for sonication (36 tubes were combined into 12 tubes) and stored on ice until processing. The sonicator was set to 60% amplitude, 2 sec on 4 sec off cycle. Each tube was individually sonicated, surrounded by ice-water in a 100 ml glass bottle, to ensure constant low temperatures for all samples. After sonication of all samples, the tubes were centrifuged for 45 min at 4000 rcf and 4° C. Supernatant was transferred to new sterile, pyrogen-free 50 ml tubes while ensuring that no significant amounts of cell fragment material was transferred. A further round of centrifugation for 45 min at 4000 rcf and 4° C. was performed to remove residual cell fragments. The resulting supernatant comprising the inventive modified bacterial hyaluronidase was directly used for HIS purification.

### 1.3.2 Purification I: HIS Affinity Chromatography, Followed by Short Term Eluate Storage (on Ice)

18 gravitational HIS-purification columns were equilibrated to general PBS downstream buffer. Contents of each centrifuged tube were distributed to 3 HIS-columns. The purification procedure was repeated twice for a single batch (2×18 columns=36 column purifications per batch). Unprocessed tubes were stored on ice at all times. Of each tube, 3×9 ml supernatant samples were carefully transferred via pipetting into 3 independent HIS-columns so as to not perturb any residual cell pellet remaining within the tube. The loaded columns were then washed by 10 ml of 10 mM imidazole PBS solution before being eluted in 6 ml 150 mM imidazole PBS solution prepared in new sterile, pyrogen-free tubes. The eluate of the 3 columns originating from the same production flask were combined (36 total purification eluates combined into 12 tubes) and diluted to 35 ml by general downstream buffer PBS. Those purified samples of the inventive modified bacterial hyaluronidase were stored on ice until the following STREP purification. The HIS-purification columns were cleaned by applying 12 ml of 300 mM imidazole PBS solution and prepared for the second run by 12 ml ultrapure water followed by 12 ml of general downstream buffer PBS.

### 1.3.3 Purification II: STREP Affinity Chromatography, Buffer Exchange (Tris-HCl NaCl), Short Term Eluate Storage (on Ice)

All tubes containing HIS-purified protein samples of the inventive modified bacterial hyaluronidase were stored on ice until completion of the entire STREP-purification process. For each combined eluate sample from HIS-purification, two iterations of syringe-based STREP-purification steps with a flowrate <5 ml/min were performed in each process (12 combined tubes from HIS-purification were processed by 24 STREP-columns). STREP-columns (5 ml bed volume) were washed and equilibrated by 2×25 ml general downstream buffer PBS. 17.5 ml of eluate sample was applied and run through the column. The loaded column was then washed by 50 ml of general downstream PBS buffer to remove any remaining contaminant proteins. By applying 20 ml of 2.5 mM d-Desthiobiotin containing PBS buffer, the protein of interest was eluted into a fresh, sterile and pyrogen-free tube, which was stored on ice at all times. The column was regenerated by 15 ml ultrapure water, followed by 15 ml 0.5 M NaOH, followed by 15 ml ultrapure water and finally by 25 ml of general downstream PBS buffer. At this stage, the next sample was loaded and purified in the same manner. A single STREP-column was used for 12 purification runs, i.e. half of the batch. After purification of the whole batch, buffer exchange was performed via Sartorius VivaSpin20 50 kDa centrifugal concentrators. Each VivaSpin was loaded by 20 ml of STREP-eluate. By centrifugation at 4000 rcf at 4° C. for 45 min, the samples were concentrated below 1 ml. Purified protein of the inventive modified bacterial hyaluronidase was collected in the VivaSpins and was buffer-exchanged by adding Tris-HCl NaCl (50 mM Tris, 154 mM NaCl, pH=7.4 at room temperature) to 20 ml. This procedure of centrifugal concentration and re-diluting to 20 ml by Tris-HCl NaCl was performed from that point on 3 times to meet specifications of the CoA. After the last centrifugation, the VivaSpins were not filled to 20 ml but to 5 ml. Each VivaSpin was then resuspended carefully and the 5 ml were taken out into a sterile, pyrogen-free tube. Two more resuspensions by 5 ml Tris-HCl NaCl were performed to ensure good recovery of the desired protein from the VivaSpins. All 12 tubes containing each 15 ml buffer exchanged protein of the inventive modified bacterial hyaluronidase were kept on ice for the polishing step.

### 1.3.4 Optional Polishing: Endotoxine (LPS Removal), Particle Removal (Filtration), Sterile Filtration, Long-Term-Storage (−15° C.)

For polishing, 2× Polymyxin B based endotoxin removal steps, 2× sterile filtration steps and 1× particle removal step were performed. In preparation, all buffer-exchanged proteins were run through sterile, pyrogen-free 0.22 μm PES filters. The filtrates were stored on ice in fresh, sterile and pyrogen-free tubes. The following steps were performed under sterile laminar flow conditions. Each sample was subject to two sequential endotoxin removal runs on (two independent) immobilized Polymyxin B columns (supplier GenScript). Each column was cleaned by a total of 15 ml of supplied regeneration buffer to remove any residual endotoxins, followed by a total of 18 ml of supplied equilibration buffer (phosphate-based) to remove all of the regeneration buffer. Additionally, a total of 15 ml of Tris-HCl NaCl was applied to further remove the phosphate buffer. The 15 ml of sterile filtered sample was loaded on the first column and gathered in a new sterile, pyrogen-free tube. To gather the remaining protein of the inventive modified bacterial hyaluronidase and enhance the yield, 5 ml of Tris-HCl/NaCl was applied to the column, and the flow-through was gathered in the same tube as well. The process was repeated using a fresh second column. Both endotoxin-removal columns were shortly regenerated multiple times within the batch run by a total of 10 ml regeneration buffer, followed by a total of 10 ml of equilibration buffer and finally followed by a total of 15 ml of Tris-HCl NaCl. A set of two endotoxin removal columns was used in total (for the whole batch). The endotoxin removal columns were regenerated after 3 sample runs. These endotoxin removal procedures were performed for all samples, and the resulting samples were stored on ice. Samples were then collected in fresh, sterile, pyrogen-free tubes and transferred under laminar flow into fresh Sartorius VivaSpin20 100 kDa centrifugal concentrators for centrifugation at 3000 rcf at 10° C. for 20 min. Under laminar flow conditions, the VivaSpin flow-through was resuspended and gathered in fresh, sterile and pyrogen-free tubes. After having gathered all the flow-through (individually for each sample), another round of sterile filtration through sterile, pyrogen-free 0.22 μm PES filters was performed inside the laminar flow. Using pyrogen-free pipette tips, a sample aliquot was made to measure the concentration of protein in the final sample via 280 nm absorption. Based on this concentration data, the combined product was diluted by Tris-HCl NaCl to a final concentration of approx. 0.1 mg/ml. Multiple small aliquots from the final product of a batch were prepared for batch analysis in sterile, pyrogen-free tubes. The final product comprising the modified bacterial hyaluronidase d016tag and all aliquoted batch analysis samples were tightly screwed and sealed by parafilm before storage at −15° C.

Protein yields [mg/ml] from E. coli BL21(DE3)/pET28a(+) shaker flask expression were determined by measuring amount of protein [mg] after HIS- & STREP purification via absorption at 280 nm (with a calculated absorption coefficient of 132590 mol−1 cm−1 and a calculated mass of 84516 Da) in reference to the culture volume [ml] during protein expression.

Thus, attained yields were approx. 0.09-0.13 mg/ml (after HIS-purification). Final yield after HIS-Purification, STREP-Purification and polishing was approx. 0.04-0.06 mg/ml.

### 1.4 Batch Analysis

### 1.4.1 Protein Purity: SDS-PAGE Analysis on Final Batch Purity by Comassie R-250 Staining

For analysis of protein purity, a single batch analysis aliquot was thawed, and the concentration was checked via 280 nm absorbance. Based on this data, samples with concentrations of 0.1 μg/25 μl up to 16 μg/25 μl were created to analyze purity on the SDS-PAGE. To each dilution 25 μl 2× LAMMELI buffer was added and gently mixed by pipetting. To improve homogeneous transmission through the gel matrix, samples were then denatured by 60° C. incubation for 20 min time before loading. A following centrifugation at 20° C. and 6000 rcf for 1 min was performed to restore condensate into the sample volume. A GenScript GelBox was filled half-way with the GenScript Tris-MOPS SDS running buffer and ready-to-use SurePage gels were installed after removal of the safety strip. The inner space was then fully filled by Tris-MOPS SDS running buffer and the combs were gently removed. Each individual loading chamber (12 per gel) was washed by pipetting 100 μl of Tris-MOPS SDS multiple times to remove glycerol. All denatured and prepared samples (50 μl) were loaded (11 per gel), and an additional chamber was used to apply 5 μl of NEB Prestained Protein ladder. The GelBox was sealed and connected to the Power Supply (120 V) until the smallest band of the ladder was close to running out of the gel (60-90 min). The power was turned off, and the gel(s) were removed from the chamber. After opening the gel cassette(s), the gels were placed inside staining boxes that were filled up to 1 cm levels with staining solution (50% MeOH, 40% ultrapure water, 10% acetic acid, 1.0 g/l Comassie R-250). Gels were stained overnight on a rocker shaker. The staining solution was removed the next morning.

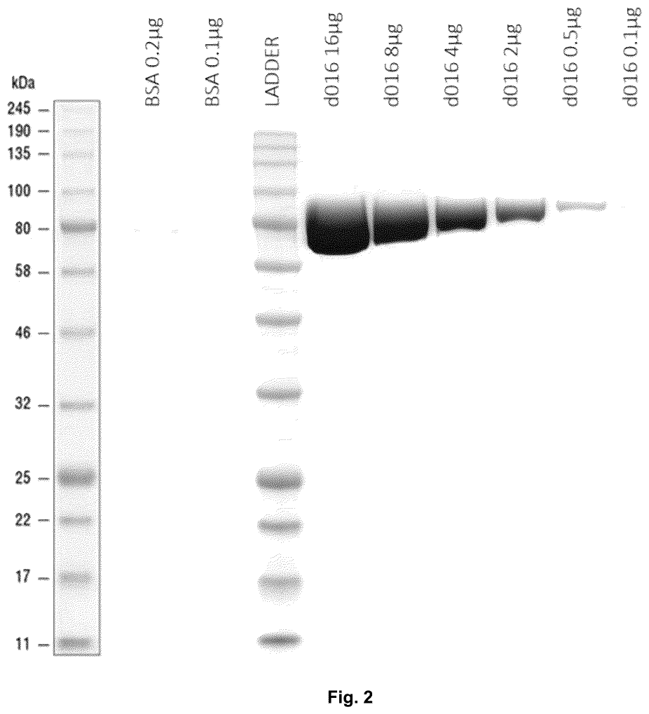

The gels were washed with demineralized water and heated in a microwave to est. 70-90° C. The gels were then transferred to the shaker for 20 min. The process was repeated until bands were clearly visible and the background signal had decreased significantly. As a final step, destaining scans (1200 dpi) were made (see FIG. 2) and purity was assessed based on the detection limit by monitoring visibility of sample contamination bands in 0.1 μg/lane up to 16 μg/lane samples. The detection limit was validated by a reference protein (BSA) of known quantity (0.2 μg and 0.1 g).

FIG. 2 represents a scan of a test batch comprising the BSA protein in concentrations 0.2 μg and 0.1 μg, ladder reference bands and the inventive modified bacterial hyaluronidase in concentrations of 16 μg, 8 μg, 4 μg, 2 μg. The protein of the inventive modified bacterial hyaluronidase migrates between the 80 kDa and 100 kDa ladder reference bands, which confirms that the inventive protein is of the correct size.

No contamination band was detectable in any of the loaded concentrations of the inventive modified bacterial hyaluronidase (0.1-16 μg), which indicates a theoretical purity of >98.8% for single contamination proteins. This finding is based on a limit-of-detection at 0.2 μg per lane, although the analyzed protein & BSA can already be detected at 0.1 μg per lane as well.

### Materials:

GenScript GenBox Mini Electrophoresis tank (L00780)
NEB Prestained Protein Ladder (P7712)
GenScript SurePage Gels 10×8 12% (M00668)
Peqlab TS-100 Thermo Shaker (see equipment)
FisherScientific Mini300V Plus Power Supply (see equipment)
Thermo Fisher Scientific NanoDrop lite (see equipment)
GenScript Tris-MOPS SDS Running buffer (M00138)
Sigma-Aldrich (GE) BSA (05470)
Sigma-Aldrich LAEMMLI 2× buffer (S3401)
Eppendorf pipettes (see equipment)
Sigma-Aldrich (Nalgene) Staining box (Z358290)
Carl-Roth Methanol (KK39.2)
Sigma-Aldrich Comassie brilliant blue R-250 (27816-25G)
Epson Workforce Pro WF-4720 (see equipment)
Sartorius Arium Mini Plus (see equipment)
IKA Rocker 2D basic Shaker

### 1.4.2 Protein Activity: Specific Activity Measurement

According to US Pharmacopoeia (USP), the unit activity of hyaluronidase is determined by calibration to the USP National Formulary Reference Standards. The unit activity was defined as: “One unit is based on the change in absorbance at 600 nm (change in turbidity) of a USP reference standard hyaluronidase which is assayed concurrently with each lot of this product.”

As this standard is no longer available for purchase, supplier Sigma-Aldrich derived a method using previously calibrated reference enzymes. The new unit definition is: “One unit will cause a change in A600 nm of 0.330 per minute at pH 5.7 at 37° C. (45-minute assay).”

And furthermore: “The change in absorbance value of 0.330 in the new unit definition was chosen in order to most closely match the results found using the USP hyaluronidase standard defined activity. As a result, the discontinued USP-based unit definition and the new unit Sigma-Aldrich unit definition will give a conversion factor of approximately 1:1 (One old unit will equal approximately one new unit).” The source on the definition of activity unit is also derivable under: https://www.sigmaaldrich.com/life-science/biochemicals/biochemical-products.html?TablePage=111679355

The turbicity assay to measure activity units for the inventive modified bacterial hyaluronidase was conducted in accordance to Sigma Aldrich protocols (which includes identical preparation of all assay buffers, reaction parameters and measurement procedures). Any changed procedures are described below:

The thus described USP-based specific activity analysis was furthermore calibrated against a “reference” hyaluronidase from Sigma-Aldrich, to remove deviations arising from individual measurements. This reference enzyme (Sigma-Aldrich bovine Hyaluronidase Typ I-S [H3506]) was used in different concentrations to allow generating a standard curve (linear fit) to convert measured transmittance (blank A600-sample A600) into U/mg. Sigma-Aldrich has determined the specific activity according to the above described protocol.

The standards (3.16 U/1.5 ml reaction—6.55 U/1.5 ml reaction) were prepared by dilution procedures. To measure the specific activity, each sample of the inventive modified bacterial hyaluronidase (syn: d016) was diluted (if necessary) into Tris-HCl NaCl to a concentration of 0.1 mg/ml. Concentrations were checked by A280 measurement, and any identified deviations were used to correct the corresponding specific activity measurement.

Subsequently, the samples were diluted 400× into enzyme dilution buffer of the Sigma-Aldrich assay—11.7 μl diluted sample was used per 1.5 ml reaction, resulting in 2.93 ng d016tag per 1.5 ml reaction (see Table 1 below). The dilution procedure for 2.93 ng enzyme was determined empirically to result within the absorbance of the standard range described above.

TABLE 1

| Sample (d016) | mg/ml | μg/μl | ng/μl |
|---|---|---|---|
| Concentration in Tris-HCl NaCl | 0.1 | 0.1 | |
| Concentration of 400x dilution into | | 0.00025 | 0.25 |
| “Enzyme Dilution Buffer” | | | |

Thus, the amount of modified bacterial hyaluronidase d016tag in final reaction (11.7 μl used for each reaction) corresponds to 2.93 ng/reaction.

For absorbance measurement, 250 μl of the 45 min reaction were combined with 1.25 ml precipitation buffer (pH=3.75) and measured for A600 in a spectrophotometer. This value was used to calculate transmittance and convert the value to specific activity according to aforementioned protocol.

Details on the turbicity method are described on the following website of Sigma Aldrich: https://www.sigmaaldrich.com/content/dam/sigma-aldrich/docs/Sigma/General Information/2/hyaluronidase.pdf

In reference to Sigma-Aldrich bovine Hyaluronidase, which has been determined according to USP Unit definition, the inventive modified bacterial hyaluronidase d016S resulted in the respective specific activity as displayed in Table 2 as follows:

TABLE 2

| | Specific | Standard |
|---|---|---|
| Sample of Test Batch | Activity (USP) | Deviation |
| 1 | 1.564.111 U/mg | 7% |
| 2 | 1.580.274 U/mg | 7% |

Thus, the general specific activity for the inventive modified bacterial hyaluronidase ranges between 1.5 Mio. USP U/mg+/−150 k USP U/mg. This equals 25.000 katal/kg+/−2.500 katal/kg.

### Materials:

Jenway 6305 Spectrophotometer (see equipment)
Sigma-Aldrich (BRAND) semi-micro UV-Cuvettes (Z628026)
Eppendorf pipettes (see equipment)
Peqlab TS-100 Thermo Shaker (see equipment)
Thermo Fisher Scientific NanoDrop lite (see equipment)
Sigma-Aldrich (Eppendorf) 3810× microtubes (Z606340)
Sigma-Aldrich (GE) BSA (05470)
Sigma-Aldrich Hyaluronic Acid (53747)
Sigma-Aldrich bovine Hyaluronidase Typ I-S(H3506)
Carl-Roth Acetic Acid (3738.2)
Carl-Roth HCl (P074.4)
Carl-Roth NaOH (9356.1)
Carl Roth Sodium Acetate (3856.1)
Carl Roth Sodium Phosphate monobasic (2370.3)
Sartorius Arium Mini Plus (see equipment)

### 1.4.3 Endotoxin Quantification

For Endotoxin quantification, the testing kit ToxinSensor™ Chromogenic LAL Endotoxin Assay Kit (L00350) by supplier GenScript was used in accordance with the manufacturer protocol. The kit included endotoxin standards, endotoxin-free water, Limulus Amebocyte Lysate, chromogenic substrate, color-stabilizers, endotoxin-free pipette tips and tubes, as well as a tube rack. All packaging were cleaned by ultrapure water and components were sterilized under laminar flow. The powder/lyophilized components were reconstituted and stored according to the manufacturer protocol. By diluting the endotoxin standard of the supplied kit, four standards for endotoxin measurement were prepared (0.1 EU/ml, 0.25 EU/ml, 0.5 EU/ml, 1.0 EU/ml). The standards were used to generate a reference curve, by which the absorption of each sample can be converted into a concentration in EU/ml. The respective standards were analyzed in parallel during all batch sample measurements. For batch analysis, a single batch analysis aliquot was thawed on ice, the tube was disinfected and washed by ultrapure water and transferred to laminar flow. 100 μl of an undiluted batch sample, 100 μl of endotoxin-free water as blank and four standards with endotoxin concentrations between 0.1 EU/ml and 1.0 EU/ml were used for a single analysis run. All sample handling was performed under sterile laminar flow conditions. The following steps were performed:

1. Incubation of well-mixed standards, blank and sample(s) with LAL for 12 min at 37° C.

2. Addition of substrate solution for chromogenic reaction and incubation at 37° C. for 6 min

3. Stepwise addition of the three color-stabilizer solutions to each measurement vial with gentle mixing

4. Transfer of the final reacted solutions to cuvettes for 545 nm absorption measurement on a spectrophotometer. Background from blank was subtracted from all batch sample measurements, and the standard curve was plotted. Endotoxin concentration of the sample(s) in EU/ml were extrapolated from the standard curve. By taking into account the final batch product/aliquot concentration of 0.1 mg/ml the endotoxin level was further described as EU/mg product.

### 1.4.4 Sterility Test

Vegetal LB-Agar (10 g/l Soya-Peptone, 10 g/l NaCl, 5 g/l Yeast Extract, 15 g/l Agar-Agar) was prepared with ultrapure water and autoclaved. The hot bottle was placed inside the ethanol-sterilized laminar flow together with sterile petri dishes. Plates were poured without addition of antibiotics to the growth media. For sterility testing, a single batch analysis aliquot was taken and thawed on ice. After the plates were solidified and cooled down, a single batch analysis aliquot was pipetted on a single plate with sterile, pyrogen-free pipette tips and spread out by sterile Lazy-L-Spreaders. A plate containing sterile filtered Tris-HCl NaCl served as control to test for environmental contaminations. A plate containing untransformed E. coli BL21(DE3) served as positive control. The plates were closed but not sealed to allow air exchange. To ensure environmental sterility, the plates for the product sterility test were kept inside the laminar flow for 4 days to check for any growth (timeframe may be adapted depending on environmental contamination speed during processing in laminar flow). No visible growth is confirmation for product sterility.

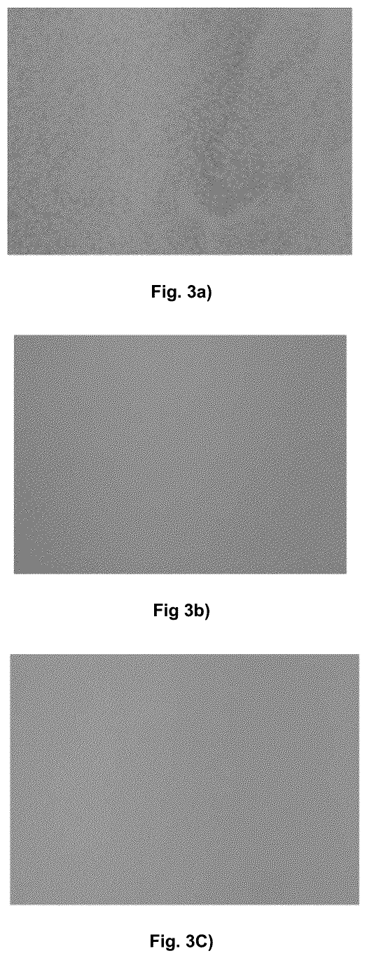

FIG. 3 show images of LB-Agar Plates comprising E. coli positive control FIG. 3a), LB-plate negative control FIGS. 3b) and d016tag sample FIG. 3c) over a time period of 4 days. The modified bacterial hyaluronidase d016tag does not show any growth in antibiotic-free, vegetal LB-Agar-Plates over a time period of 4 days and, thus, is to be regarded sterile.

Accordingly, the modified bacterial hyaluronidase d016tag can be used for pharmaceutical compositions, in particular for pharmaceutical compositions requiring a sterile quality, such as intravenous application.

### Materials:

Carl Roth vegetal Peptone (2832.2)
Carl Roth NaCl (3957.3)
Carl Roth Yeast Extract (2363.3)
Carl Roth Agar-Agar (6494.1)
Sigma Aldrich (sterile) petri dish (P5981)
Sigma-Aldrich (sterile) Lazy-L-Spreaders (Z376779)
Carl Roth (Sorenson Bioscience) pyrogen-free 1000 μl filter-tips (9773.1)
Eppendorf pipettes (see equipment)

### 1.4.5 Stability & Solubility, Freeze-Thaw Tests

To test stability & solubility of inventive samples, specific activity measurements of multiple concentrations of purified protein were conducted over time. Multiple sample dilutions were prepared in sterile-filtered Tris-HCl NaCl at 0.2 mg/ml (200% final stock solution of product), 0.1 mg/ml (targeted final stock solution of product) and 0.01 mg/ml (low concentration dose for targeted application). Inventive protein concentrations were measured using 280 nm absorption. Respective dilutions were stored at −15° C., 2-8° C. and 25° C. representing relevant storage and handling conditions. Aliquots for Specific Activity measurements were taken at the start of stability tests, after 1 day, 2 days, 3 days, 1 week, 2 weeks and 4 weeks. All aliquots were stored in Tris-HCl NaCl with 154 mM NaCl concentration, which equals the ionic strength of 0.9% medicinal saline solution. Aliquots were stored on ice until measurement. Measurements were performed according to the activity measurement protocol.

Furthermore, freeze-thaw tests were conducted to benchmark how product solution withstands ice crystal formation during freeze-thaw processes. Samples were frozen 5× at −80° C. and thawed again, while aliquots were taken after each cycle to measure activity. General stability & solubility results demonstrated that Specific Activity overall does not drop within 7 days if the sample concentration is above 0.1 mg/ml for −15° C., 2-8° C. and 25° C. The freeze-thaw stability of a −80° C. freeze-thaw cycle does not reduce Specific Activity if the sample is frozen less than 2 times. Sample storage concentration showed loss of protein, most likely due to adsorption, within a single freeze-thaw cycle and within a single day of storage at −15° C. and 2-8° C. of 5-15%. At 25° C. samples with a concentration above 0.2 mg/ml seem to show no concentration loss over 7 days.

The results indicate that the final product should preferably be stored in protein-low-bind tubes, at concentrations above 0.2 mg/ml and frozen not more than once to ensure absolute stability. The measurements have been conducted in small microtubes (1.5 ml) with a low filling volume of 150-1500 μl. This results in a much smaller volume-to-surface quotient, compared to product storage in 50 ml tubes. Therefore, the results above are very likely to be more dependent on adsorption effects than a commercialized product.

### Materials:

Sigma-Aldrich Tris (TRIS-RO)
Carl-Roth HCl (P074.4)
Carl Roth NaCl (3957.3)
Thermo Fisher Scientific NanoDrop lite (see equipment)
Sigma-Aldrich (Eppendorf) 3810× microtubes (Z606340)
Eppendorf pipettes (see equipment)
   2: Comparison of Specific Activity Between Prior Art Hyaluronidase Peptides and Inventive Modified Bacterial Hyaluronidase (Syn: d016)
   2.1 Comparison of Specific Activity Between Bacterial Hyaluronidase Derived from Streptomyces Koganeiensis (Syn: Messina Hyaluronidase) and Inventive Modified Bacterial Hyaluronidase

“The rHyal_Sk in the periplasmic soluble portion was thus produced at a final concentration of approximately 2 g/L of culture medium with very high functional activity (more than 40 000 units/mg), 670- to 750-fold higher than the autologous Hyal produced by fermentation.” (see Messina et al., “Identification and characterization of a bacterial hyaluronidase and its production in recombinant form”, Federation of European Biochemical Societies (FEBS) Letters, Volume 590, Issue 14, July 2016, pp. 2180-2189)

Accordingly, the Specific Activity per mg for the bacterial Messina hyaluronidase derived from Streptomyces koganeiensis is: >40 000 U/mg, i.e. between 40 000 U/mg and 50 000 U/mg.

Specific Activity of modified bacterial hyaluronidase d016tag as set out in Example 1.4.2 is: 1 500 000 U/mg.

Thus, the Specific Activity of the modified bacterial hyaluronidase d016tag per mg protein is appr. 30× to 37.5× of the Specific Activity of the comparative Messina hyaluronidase.

2.2 Comparison of Human PH20 Hyaluronidase and Inventive Modified Bacterial Hyaluronidase d016

“The activity of Human PH20, His Tag (Cat. No. PH0-H5225) is measured by its ability to hydrolyze HA in turbidimetric assay (45 minute assay). The specific activity is >40,000 U/mg. (Unit Definition: One unit of Hyaluronidase activity will cause a change in A600 of 0.330 per minute at pH 5.35 at 37° C. in a 2.0 mL reaction mixture)”-ACRObiosystems, https://www.acrobiosystems.com/P563-Human-PH20--SPAM1-Protein-His-Tag.html

Accordingly, the Specific Activity per mg for the human PH20 hyaluronidase is: >40 000 U/mg, i.e. between 40 000 U/mg and 50 000 U/mg.

Specific Activity of modified bacterial hyaluronidase d016tag as set out in Example 1.4.2 is: 1 500 000 U/mg.

Thus, the Specific Activity of the modified bacterial hyaluronidase d016tag per mg protein is appr. 30× to 37.5× of the Specific Activity of the comparative human PH20 hyaluronidase.

2.3 Comparison of Bovine Hyaluronidase and Inventive Modified Bacterial Hyaluronidase d016

“Hyaluronidase degrades hyaluronan and has been found to be inappropriately regulated during cancer progression. These enzymes randomly cleave β-N-acetylhexosamine-[1→4] glycosidic bonds in hyaluronic acid, chondroitin, and chondroitin sulfates. Unit Definition: One unit will cause a change in % transmittance at 600 nm of 0.330 per minute at pH 5.35 at 37° C. in a 2.0 mL reaction mixture (45 minute assay).”—Sigma-Aldrich, Supplier of Hyaluronidase from bovine testes

Hyaluronidase from bovine testes, Type I-S, lyophilized powder 400-1 000 U/mg solid

Hyaluronidase from bovine testes, Type IV-S, powder, suitable for mouse embryo cell culture 750-3 000 U/mg solid,

Hyaluronidase from bovine testes, Type IV-S, lyophilized powder (essentially salt-free), 750-3 000 U/mg,

Hyaluronidase from bovine testes, Type VIII, lyophilized powder, 300-1 000 U mg,

Hyaluronidase from bovine testes, Type VI-S, lyophilized powder, 3 000-15 000 U/mg.

Accordingly, the Specific Activity of bovine hyaluronidases widely range between 300 and 15 000 U/mg.

Specific Activity of modified bacterial hyaluronidase d016tag as set out in Example 1.4.2 is: 1 500 000 U/mg.

Thus, the Specific Activity of the modified bacterial hyaluronidase d016tag per mg protein is appr. 100× to 5 000× of the Specific Activity of the comparative bovine hyaluronidases.

### FURTHER EMBODIMENTS OF THE PRESENT INVENTION

Embodiment 1 (first inventive aspect): A process of production of a purified modified bacterial hyaluronidase polypeptide comprising or consisting of the following steps:
a. Culturing a transformed host cell with recombinant expression vector comprising or consisting of a nucleic acid encoding a modified bacterial hyaluronidase polypeptide in a suitable growth medium under suitable growth conditions to express the bacterial hyaluronidase polypeptide comprising or consisting of at least 90% sequence identity to SEQ ID No. 1, wherein the hyaluronidase polypeptide comprises a C-terminal tag and an N-terminal tag,
b. Harvesting the cultured transformed host cell of step a),
c. Lysing the harvested host cells of step b) and separating resulting host cell fragments from resulting host cell content comprising the bacterial hyaluronidase polypeptide, and
d. Purifying the resulting host cell content of step c) with a first affinity chromatography corresponding to the C-terminal tag and a second affinity chromatography corresponding to the N-terminal tag to result in a purified form of the bacterial hyaluronidase polypeptide comprising or consisting of at least 90% sequence identity to SEQ ID No. 1, wherein the hyaluronidase polypeptide comprises the C-terminal tag and the N-terminal tag, and
e. Removing at least part of the C-terminal tag of the purified bacterial hyaluronidase of step d) and/or removing at least part of the N-terminal tag of the purified bacterial hyaluronidase of step d).

Embodiment 2: The process of production of a purified bacterial hyaluronidase polypeptide according to embodiment 1, wherein the host cell is E. coli, preferably E. coli BL21(DE3) competent cells.

Embodiment 3: The process of production of a purified bacterial hyaluronidase polypeptide according to embodiment 1 or 2, wherein the C-terminal tag is a HIS tag, preferably a HIS tag of SEQ ID No. 7, and/or wherein the N-terminal tag is a STREP tag, preferably a STREP tag of SEQ ID No. 5.

Embodiment 4 (second inventive aspect): A modified bacterial hyaluronidase polypeptide comprising or consisting of at least 90% sequence identity to SEQ ID No. 9 or SEQ ID No. 10 obtainable according to the production process according to any one of embodiments 1 to 3, wherein the modified bacterial hyaluronidase polypeptide may respectively comprise no, one or two remaining residues of the C-terminal tag and/or no, one or two remaining residues of the N-terminal tag.

Embodiment 5 (third inventive aspect): A pharmaceutical composition comprising the modified bacterial hyaluronidase polypeptide according to embodiment 4 in a therapeutically effective amount and one or more pharmaceutically acceptable excipients, wherein the pharmaceutical composition is used in the treatment or prophylaxis of a hyaluronan-associated and/or proteoglycan-associated disease or disorder, preferably selected from a group consisting homozygous familial hypercholesterolemia (xanthomatose), heterozygous familial hypercholesterolemia, diabetic foot syndrome, arterial hypertension or cardiac insufficiency.

Embodiment 6: The pharmaceutical composition according to embodiment 5, wherein a unit dose of the composition comprises the modified bacterial hyaluronidase polypeptide in an amount of 200 U per kg/per day to 30,000 U per kg/per day.

Embodiment 7: The pharmaceutical composition according to embodiment 5 or 6, wherein the composition is selected from a solid, semi-solid or liquid application form.

Embodiment 8: The pharmaceutical composition according to any one of embodiments 5 to 7, wherein the composition is suitable for per oral, nasal, transdermal, rectal, intravenous, or intramuscular application.

Embodiment 9 (fourth inventive aspect): A method of treating a hyaluronan-associated and/or proteoglycan-associated disease or disorder comprising or consisting of administering the modified bacterial hyaluronidase polypeptide according to embodiment 4, or the pharmaceutical composition according to embodiments 5 to 8 to a subject in need thereof.

Embodiment 10: The method of treatment according to embodiment 9, wherein the hyaluronan-associated and/or proteoglycan-associated disease or disorder is selected from a group consisting of a homozygous familial hypercholesterolemia (xanthomatose), heterozygous familial hypercholesterolemia, diabetic foot syndrome, arterial hypertension or cardiac insufficiency.

1-14. (canceled)

15. A process of production of a purified modified bacterial hyaluronidase polypeptide comprising the following steps:
a. Culturing a transformed host cell with recombinant expression vector comprising a nucleic acid encoding a modified bacterial hyaluronidase polypeptide in a suitable growth medium under suitable growth conditions to express the bacterial hyaluronidase polypeptide comprising at least 90% sequence identity to SEQ ID No. 1, wherein the hyaluronidase polypeptide comprises a C-terminal tag and an N-terminal tag,
b. Harvesting the cultured transformed host cell of step a),
c. Lysing the harvested host cells of step b) and separating resulting host cell fragments from resulting host cell content comprising the bacterial hyaluronidase polypeptide,
d. Purifying the resulting host cell content of step c) with a first affinity chromatography corresponding to the C-terminal tag and a second affinity chromatography corresponding to the N-terminal tag to result in a purified form of the bacterial hyaluronidase polypeptide comprising or consisting of at least 90% sequence identity to SEQ ID No. 1, wherein the hyaluronidase polypeptide comprises the C-terminal tag and the N-terminal tag, and
e. Removing at least part of the C-terminal tag of the purified bacterial hyaluronidase of step d) and/or removing at least part of the N-terminal tag of the purified bacterial hyaluronidase of step d).

16. The process of production of a purified bacterial hyaluronidase polypeptide according to claim 15, wherein the host cell is E. coli, preferably E. coli BL21(DE3) competent cells.

17. The process of production of a purified bacterial hyaluronidase polypeptide according to claim 15, wherein the C-terminal tag is removed using a Tobacco Etch Virus (TEV) protease cleaving site comprising the SEQ ID No. 16 in the bacterial hyaluronidase amino acid sequence.

18. A modified bacterial hyaluronidase polypeptide comprising 99.5%, 99.6, 99.7, 99.8, 99.9, or 100% sequence identity to any one of SEQ ID Nos. 9, 10, 20, 22, 24, 26, 28, 30, 32, 34, 36, or 38.

19. A pharmaceutical composition comprising the modified bacterial hyaluronidase polypeptide according to claim 4 in a therapeutically effective amount and one or more pharmaceutically acceptable excipients, wherein the pharmaceutical composition is used in the treatment or prophylaxis of a hyaluronan-associated and/or proteoglycan-associated disease or disorder, preferably selected from a group consisting homozygous familial hypercholesterolemia (xanthomatose), heterozygous familial hypercholesterolemia, diabetic foot syndrome, arterial hypertension or cardiac insufficiency.

20. The pharmaceutical composition according to claim 19, wherein a unit dose of the composition comprises the modified bacterial hyaluronidase polypeptide in an amount of 200 U per kg/per day to 30,000 U per kg/per day.

21. The pharmaceutical composition according to claim 19, wherein the composition is selected from a solid, semi-solid or liquid application form.

22. The pharmaceutical composition according to claim 19, wherein the composition is suitable for per oral, nasal, transdermal, rectal, intravenous, or intramuscular application.

23. A method of treating a hyaluronan-associated and/or proteoglycan-associated disease or disorder comprising administering the modified bacterial hyaluronidase polypeptide according to claim 18 to a subject in need thereof.

24. The method of treatment according to claim 23, wherein the hyaluronan-associated and/or proteoglycan-associated disease or disorder is selected from a group consisting of a homozygous familial hypercholesterolemia (xanthomatose), heterozygous familial hypercholesterolemia, diabetic foot syndrome, arterial hypertension and cardiac insufficiency.

25. A process of production of a purified modified bacterial hyaluronidase polypeptide of at least 90% sequence identity of any one of SEQ ID Nos. 9, 10, 30, 32, 34, 36, or 38 comprising the following steps:
a. Culturing a transformed host cell with recombinant expression vector comprising or consisting of a nucleic acid of at least 90% sequence identity of any one of SEQ ID Nos. 29, 31, 33, 35, or 37 encoding a modified bacterial hyaluronidase polypeptide in a suitable growth medium under suitable growth conditions to express the bacterial hyaluronidase polypeptide, wherein the bacterial hyaluronidase polypeptide comprises a N-terminal tag including at least a first N-terminal purification tag connected by a cleavage site to a second different purification tag which is connected to the hyaluronidase polypeptide either directly or by a second cleavage site,
b. Harvesting the cultured transformed host cell of step a),
c. Lysing the harvested host cells of step b) and separating resulting host cell fragments from resulting host cell content comprising the bacterial hyaluronidase polypeptide, and
d. Purifying the resulting host cell content of step c) with a first affinity chromatography corresponding to the N-terminal first purification tag, removing the first purification tag from the remaining hyaluronidase sequence and purifying the resulting hyaluronidase sequence with a second affinity chromatography corresponding to the second purification tag and optionally removing the N-terminal second purification tag to result in a purified form of the bacterial hyaluronidase polypeptide comprising or consisting of at least 90% sequence identity to SEQ ID Nos. 9, 10, 30, 32, 34, 36, or 38.

26. The process of production of a purified bacterial hyaluronidase polypeptide according to claim 25, wherein the host cell is E. coli, preferably E. coli BL21(DE3) competent cells.

27. The process of production of a purified bacterial hyaluronidase polypeptide according to claim 25, wherein the purification tags are selected from a HIS tag and Strep tag, in particular wherein the first purification N-terminal tag is a HIS tag, preferably a HIS tag of SEQ ID No. 7, and/or wherein the N-terminal second purification tag is a STREP tag, preferably a STREP tag of SEQ ID No. 5.

28. The process of production of a purified bacterial hyaluronidase polypeptide according to claim 15, wherein the purification tags are removed from the hyaluronidase polypeptide using a protease, such as a TEV-protease, or an enteropeptidase.

29. A method of treating a hyaluronan-associated and/or proteoglycan-associated disease or disorder comprising administering the pharmaceutical composition according to claim 19 to a subject in need thereof.

30. A method of treating a hyaluronan-associated and/or proteoglycan-associated disease or disorder comprising administering the pharmaceutical composition according to claim 20 to a subject in need thereof.

31. A method of treating a hyaluronan-associated and/or proteoglycan-associated disease or disorder comprising administering the pharmaceutical composition according to claim 21 to a subject in need thereof.

32. A method of treating a hyaluronan-associated and/or proteoglycan-associated disease or disorder comprising administering the pharmaceutical composition according to claim 22 to a subject in need thereof.

33. The process of production of a purified bacterial hyaluronidase polypeptide according to claim 25, wherein the purification tags are removed from the hyaluronidase polypeptide using a protease, such as a TEV-protease, or an enteropeptidase.
